SEQUENCE LISTING (90 sequences)

SEQ ID NO: 1 - DNA, 24 bp, synthetic construct
  misc_feature 1..24: note = forward primer
  source 1..24: mol_type = other DNA
ccctgtttgtggctgctataattt

SEQ ID NO: 2 - DNA, 22 bp, synthetic construct
  misc_feature 1..22: note = reverse primer
  source 1..22: mol_type = other DNA
gggaagaggaaggtgtcatctg

SEQ ID NO: 3 - DNA, 21 bp, synthetic construct
  misc_feature 1..21: note = forward primer
  source 1..21: mol_type = other DNA
gctgcggaaacttcaggaaat

SEQ ID NO: 4 - DNA, 23 bp, synthetic construct
  misc_feature 1..23: note = reverse primer
  source 1..23: mol_type = other DNA
agagacgtgtcactcctggactt

SEQ ID NO: 5 - DNA, 24 bp, synthetic construct
  misc_feature 1..24: note = forward primer
  source 1..24: mol_type = other DNA
acgtcagtacaaggagatgttgga

SEQ ID NO: 6 - DNA, 26 bp, synthetic construct
  misc_feature 1..26: note = reverse primer
  source 1..26: mol_type = other DNA
atcctattgcagaatgcttctttacc

SEQ ID NO: 7 - DNA, 20 bp, synthetic construct
  misc_feature 1..20: note = forward primer
  source 1..20: mol_type = other DNA
agggtctgggccatagaact

SEQ ID NO: 8 - DNA, 21 bp, synthetic construct
  misc_feature 1..21: note = reverse primer
  source 1..21: mol_type = other DNA
ccaccacgctcttctgtctac

SEQ ID NO: 9 - DNA, 20 bp, synthetic construct
  misc_feature 1..20: note = forward primer
  source 1..20: mol_type = other DNA
agttgccttcttgggactga

SEQ ID NO: 10 - DNA, 20 bp, synthetic construct
  misc_feature 1..20: note = reverse primer
  source 1..20: mol_type = other DNA
tccacgatttcccagagaac

SEQ ID NO: 11 - DNA, 20 bp, synthetic construct
  misc_feature 1..20: note = forward primer
  source 1..20: mol_type = other DNA
ccagaagaaaatgaggtcgg

SEQ ID NO: 12 - DNA, 19 bp, synthetic construct
  misc_feature 1..19: note = reverse primer
  source 1..19: mol_type = other DNA
agcgctcaaggagaagacc

SEQ ID NO: 13 - DNA, 20 bp, synthetic construct
  misc_feature 1..20: note = forward primer
  source 1..20: mol_type = other DNA
cataagctgggcaagtggta

SEQ ID NO: 14 - DNA, 20 bp, synthetic construct
  misc_feature 1..20: note = reverse primer
  source 1..20: mol_type = other DNA
ggatgtacagatgggggatg

SEQ ID NO: 15 - DNA, 25 bp, synthetic construct
  misc_feature 1..25: note = forward primer
  source 1..25: mol_type = other DNA
agatctccagttcttacacgaccac

SEQ ID NO: 16 - DNA, 22 bp, synthetic construct
  misc_feature 1..22: note = reverse primer
  source 1..22: mol_type = other DNA
gacggatgtcttcttccaggtg

SEQ ID NO: 17 - DNA, 20 bp, synthetic construct
  misc_feature 1..20: note = forward primer
  source 1..20: mol_type = other DNA
gatgaaccatctccgttggc

SEQ ID NO: 18 - DNA, 22 bp, synthetic construct
  misc_feature 1..22: note = reverse primer
  source 1..22: mol_type = other DNA
cccaattatgaatcgggagtgc

SEQ ID NO: 19 - DNA, 23 bp, synthetic construct
  misc_feature 1..23: note = forward primer
  source 1..23: mol_type = other DNA
gaaccactgctttcaaaaactgg

SEQ ID NO: 20 - DNA, 20 bp, synthetic construct
  misc_feature 1..20: note = reverse primer
  source 1..20: mol_type = other DNA
tgctgttctttgccacgtca

SEQ ID NO: 21 - DNA, 20 bp, synthetic construct
  misc_feature 1..20: note = forward primer
  source 1..20: mol_type = other DNA
tgaaagaagtgggagtgggc

SEQ ID NO: 22 - DNA, 20 bp, synthetic construct
  misc_feature 1..20: note = reverse primer
  source 1..20: mol_type = other DNA
cgaattccacgcccagtttg

SEQ ID NO: 23 - DNA, 21 bp, synthetic construct
  misc_feature 1..21: note = forward primer
  source 1..21: mol_type = other DNA
ctcagtgggagcgactcttca

SEQ ID NO: 24 - DNA, 21 bp, synthetic construct
  misc_feature 1..21: note = reverse primer
  source 1..21: mol_type = other DNA
ggcctctgtggtacacgacaa

SEQ ID NO: 25 - DNA, 19 bp, synthetic construct
  misc_feature 1..19: note = forward primer
  source 1..19: mol_type = other DNA
gcagcgctgtgtggtcatc

SEQ ID NO: 26 - DNA, 24 bp, synthetic construct
  misc_feature 1..24: note = reverse primer
  source 1..24: mol_type = other DNA
catccatttgtcactcaggaactt

SEQ ID NO: 27 - DNA, 20 bp, synthetic construct
  misc_feature 1..20: note = forward primer
  source 1..20: mol_type = other DNA
ggcttcctgcctagtttcca

SEQ ID NO: 28 - DNA, 21 bp, synthetic construct
  misc_feature 1..21: note = reverse primer
  source 1..21: mol_type = other DNA
agtcaaaggcatccaccaaag

SEQ ID NO: 29 - DNA, 20 bp, synthetic construct
  misc_feature 1..20: note = forward primer
  source 1..20: mol_type = other DNA
ctatgggatcagccagaaag

SEQ ID NO: 30 - DNA, 20 bp, synthetic construct
  misc_feature 1..20: note = reverse primer
  source 1..20: mol_type = other DNA
atgaggttggctttcaggag

SEQ ID NO: 31 - DNA, 22 bp, synthetic construct
  misc_feature 1..22: note = forward primer
  source 1..22: mol_type = other DNA
ccccaaagccctcagtcgttgt

SEQ ID NO: 32 - DNA, 20 bp, synthetic construct
  misc_feature 1..20: note = reverse primer
  source 1..20: mol_type = other DNA
cagccactcaggcaccgcac

SEQ ID NO: 33 - DNA, 20 bp, synthetic construct
  misc_feature 1..20: note = forward primer
  source 1..20: mol_type = other DNA
aaggcagtggagcaggtgaa

SEQ ID NO: 34 - DNA, 20 bp, synthetic construct
  misc_feature 1..20: note = reverse primer
  source 1..20: mol_type = other DNA
ccagcagactcaatacacac

SEQ ID NO: 35 - DNA, 22 bp, synthetic construct
  misc_feature 1..22: note = forward primer
  source 1..22: mol_type = other DNA
cgtgtatgaaagaagctgatgc

SEQ ID NO: 36 - DNA, 22 bp, synthetic construct
  misc_feature 1..22: note = reverse primer
  source 1..22: mol_type = other DNA
acgaaatccattttcttctgga

SEQ ID NO: 37 - DNA, 24 bp, synthetic construct
  misc_feature 1..24: note = forward primer
  source 1..24: mol_type = other DNA
atgccaggacaccctgtgaacttc

SEQ ID NO: 38 - DNA, 24 bp, synthetic construct
  misc_feature 1..24: note = reverse primer
  source 1..24: mol_type = other DNA
acatcattgcagaagtagcagcca

SEQ ID NO: 39 - DNA, 22 bp, synthetic construct
  misc_feature 1..22: note = forward primer
  source 1..22: mol_type = other DNA
acagaagatggatcgcgttatg

SEQ ID NO: 40 - DNA, 19 bp, synthetic construct
  misc_feature 1..19: note = reverse primer
  source 1..19: mol_type = other DNA
ccccgtgactcactgaagg

SEQ ID NO: 41 - DNA, 23 bp, synthetic construct
  misc_feature 1..23: note = forward primer
  source 1..23: mol_type = other DNA
tccaggagagatgctcacacttt

SEQ ID NO: 42 - DNA, 20 bp, synthetic construct
  misc_feature 1..20: note = reverse primers
  source 1..20: mol_type = other DNA
tgcgtggaacaggtttgaaa

SEQ ID NO: 43 - DNA, 18 bp, synthetic construct
  misc_feature 1..18: note = forward primer
  source 1..18: mol_type = other DNA
gacccgagaaggcgcttt

SEQ ID NO: 44 - DNA, 22 bp, synthetic construct
  misc_feature 1..22: note = reverse primer
  source 1..22: mol_type = other DNA
tttgccaacccaaatgtagaaa

SEQ ID NO: 45 - DNA, 20 bp, synthetic construct
  misc_feature 1..20: note = forward primer
  source 1..20: mol_type = other DNA
atcgacttcagcccgtacct

SEQ ID NO: 46 - DNA, 20 bp, synthetic construct
  misc_feature 1..20: note = reverse primer
  source 1..20: mol_type = other DNA
ccgtagtcgtcggagaagag

SEQ ID NO: 47 - DNA, 21 bp, synthetic construct
  misc_feature 1..21: note = forward primer
  source 1..21: mol_type = other DNA
agtccctgccctttgtacaca

SEQ ID NO: 48 - DNA, 19 bp, synthetic construct
  misc_feature 1..19: note = reverse primer
  source 1..19: mol_type = other DNA
cgatccgagggcctcacta

SEQ ID NO: 49 - DNA, 20 bp, synthetic construct
  source 1..20: mol_type = other DNA
aagggagagaaaggagatgc

SEQ ID NO: 50 - DNA, 20 bp, synthetic construct
  source 1..20: mol_type = other DNA
tacacataagcggcttctcc

SEQ ID NO: 51 - DNA, 20 bp, synthetic construct
  source 1..20: mol_type = other DNA
attgggatcatcttgctggt

SEQ ID NO: 52 - DNA, 19 bp, synthetic construct
  source 1..19: mol_type = other DNA
cctgctgttcacagttgcc

SEQ ID NO: 53 - DNA, 20 bp, synthetic construct
  source 1..20: mol_type = other DNA
cgaggattctctggaactgc

SEQ ID NO: 54 - DNA, 21 bp, synthetic construct
  source 1..21: mol_type = other DNA
ggcctctgtggtacacgacaa

SEQ ID NO: 55 - DNA, 18 bp, synthetic construct
  source 1..18: mol_type = other DNA
agagtggaggcgcatgct

SEQ ID NO: 56 - DNA, 22 bp, synthetic construct
  source 1..22: mol_type = other DNA
ggcatctaggaggaagctgttc

SEQ ID NO: 57 - DNA, 20 bp, synthetic construct
  source 1..20: mol_type = other DNA
ctacgtcatccagagtggca

SEQ ID NO: 58 - DNA, 20 bp, synthetic construct
  source 1..20: mol_type = other DNA
ctgttcatcatagcgctcca

SEQ ID NO: 59 - DNA, 23 bp, synthetic construct
  source 1..23: mol_type = other DNA
cctctaggtttctttgccaacag

SEQ ID NO: 60 - DNA, 19 bp, synthetic construct
  source 1..19: mol_type = other DNA
aagctgcaggcctcaggat

SEQ ID NO: 61 - DNA, 20 bp, synthetic construct
  source 1..20: mol_type = other DNA
aaaagtgcctgaaaccagag

SEQ ID NO: 62 - DNA, 20 bp, synthetic construct
  source 1..20: mol_type = other DNA
tcacctcctgctctaaaagg

SEQ ID NO: 63 - DNA, 23 bp, synthetic construct
  source 1..23: mol_type = other DNA
tcctggaactaagtggacgcaag

SEQ ID NO: 64 - DNA, 23 bp, synthetic construct
  source 1..23: mol_type = other DNA
cagacacactcctgcgcatagac

SEQ ID NO: 65 - DNA, 20 bp, synthetic construct
  source 1..20: mol_type = other DNA
ctccatctgctggccttctc

SEQ ID NO: 66 - DNA, 21 bp, synthetic construct
  source 1..21: mol_type = other DNA
catccaggctctctggcttct

SEQ ID NO: 67 - DNA, 22 bp, synthetic construct
  source 1..22: mol_type = other DNA
agccgtgaccactgacaacgag

SEQ ID NO: 68 - DNA, 23 bp, synthetic construct
  source 1..23: mol_type = other DNA
gctgcatggttctgagtgctaag

SEQ ID NO: 69 - DNA, 20 bp, synthetic construct
  source 1..20: mol_type = other DNA
cacaatgccatcaggtttgg

SEQ ID NO: 70 - DNA, 23 bp, synthetic construct
  source 1..23: mol_type = other DNA
gctggtcgatatcactggagatc

SEQ ID NO: 71 - DNA, 19 bp, synthetic construct
  source 1..19: mol_type = other DNA
cagcacggtgaagccattc

SEQ ID NO: 72 - DNA, 17 bp, synthetic construct
  source 1..17: mol_type = other DNA
gcgtgcatccgcttgtg

SEQ ID NO: 73 - DNA, 22 bp, synthetic construct
  source 1..22: mol_type = other DNA
cacccagatgcaaaactttcag

SEQ ID NO: 74 - DNA, 23 bp, synthetic construct
  source 1..23: mol_type = other DNA
ctgctctttatacttgctcacag

SEQ ID NO: 75 - DNA, 19 bp, synthetic construct
  source 1..19: mol_type = other DNA
accctgtcatcccacagag

SEQ ID NO: 76 - DNA, 22 bp, synthetic construct
  source 1..22: mol_type = other DNA
tgtttggtggagtcctaaggtc

SEQ ID NO: 77 - DNA, 21 bp, synthetic construct
  source 1..21: mol_type = other DNA
actgccacacctccagtcatt

SEQ ID NO: 78 - DNA, 21 bp, synthetic construct
  source 1..21: mol_type = other DNA
ctttgcctcactcaggattgg

SEQ ID NO: 79 - DNA, 20 bp, synthetic construct
  source 1..20: mol_type = other DNA
gacggctatggtgtgtcgta

SEQ ID NO: 80 - DNA, 19 bp, synthetic construct
  source 1..19: mol_type = other DNA
tccagcccagcacatgaac

SEQ ID NO: 81 - DNA, 20 bp, synthetic construct
  source 1..20: mol_type = other DNA
agcagcaagtgtcccaaaga

SEQ ID NO: 82 - DNA, 20 bp, synthetic construct
  source 1..20: mol_type = other DNA
ggtggtccatggaatcctga

SEQ ID NO: 83 - DNA, 22 bp, synthetic construct
  source 1..22: mol_type = other DNA
caagtgtgtcagcgcctatgct

SEQ ID NO: 84 - DNA, 22 bp, synthetic construct
  source 1..22: mol_type = other DNA
caagtgtgtcagcgcctatgct

SEQ ID NO: 85 - DNA, 18 bp, synthetic construct
  source 1..18: mol_type = other DNA
tgccagaaacagcatcag

SEQ ID NO: 86 - DNA, 19 bp, synthetic construct
  source 1..19: mol_type = other DNA
caaattccgcagtgtctct

SEQ ID NO: 87 - DNA, 20 bp, synthetic construct
  source 1..20: mol_type = other DNA
tgcgtggcctttgaaatgtg

SEQ ID NO: 88 - DNA, 20 bp, synthetic construct
  source 1..20: mol_type = other DNA
ctccatgtccgtgaactgct

SEQ ID NO: 89 - DNA, 20 bp, synthetic construct
  source 1..20: mol_type = other DNA
atggcagcatctacaaccct

SEQ ID NO: 90 - DNA, 20 bp, synthetic construct
  source 1..20: mol_type = other DNA
aggactcgtttgtacccgtt

(19) United States
(12) Patent Application Publication
(10) Pub. No.: US 2025/0268855 A1
(43) Pub. Date: Aug. 28, 2025

(54) PHARMACEUTICAL AND NUTRACEUTICAL COMPOSITIONS WITH A COMBINATION OF AMINO ACIDS AND ITS USE IN DISEASES CHARACTERIZED BY LIPID ACCUMULATION IN TISSUES

(71) Applicants: FUNDACIÓ EURECAT, CERDANYOLA DEL VALLÈS (ES); FUNDACIÓ INSTITUT D'INVESTIGACIÓ BIOMÈDICA DE GIRONA DR. JOSEP TRUETA, SALT (ES); CONSORCIO CENTRO DE INVESTIGACIÓN BIOMÉDICA EN RED, MADRID (ES)

(72) Inventors: JOSEP MARIA DEL BAS PRIOR, TARRAGONA (ES); XAVIER ESCOTÉ MIRÓ, L'ESPLUGA DEFRANCOLÍ (ES); SERGIO QUESADA VÁZQUEZ, TARRAGONA (ES); JOSÉ MANUEL FERNÁNDEZ-REAL LEMOS, GIRONA (ES); JORDI MAYNERIS PERXACHS, FONTCOBERTA (VEÏNAT DE MELIANTA) (ES); JULIO CARLOS BAUDIN LUQUE, REUS (ES); ANTONI CAIMARI PALOU, REUS (ES); FRANCESC PUIGGRÒS LLAVINÉS, REUS (ES); ANA CASTELLS NOBAU, SALT (ES)

(21) Appl. No.: 18/859,892

(22) PCT Filed: Apr. 25, 2023
(86) PCT No.: PCT/EP2023/060827

§ 371 Date: Oct. 24, 2024

(30) Foreign Application Priority Data
Apr. 25, 2022 (EP) 22382390.7

(51) Int. Cl. A61K 31/198; A23L 2/52; A61K 31/4172; A61P 1/16; A61P 3/04

(52) U.S. Cl. CPC A61K 31/198; A23L 2/52; A61K 31/4172; A61P 1/16; A61P 3/04

(57) ABSTRACT

A pharmaceutical or dietary supplement composition including a combination of compounds including histidine, serine, cysteine and carnosine. The compositions can be ingredients in food products, and they have been proved effective in the treatment of diseases characterized by accumulation of fat in tissues, such as in fatty liver disease, obesity, and atherosclerosis.

This application claims the benefit of European Patent Application 22382390.7 filed Apr. 25 2022.

### TECHNICAL FIELD

Present invention relates to the field of dietetical supplements and pharmaceutical compositions to reduce the risk or to treat diseases related with fat tissue deposition. Thus, it relates to the field of nutrition and medicine.

### BACKGROUND ART

Due to the increased and worldwide extended consumption of a diet know as Western diet, including processed food and food rich in fat, diseases or disorders related with the deposition of fat tissue within several parts of the body, including viscera, blood vessels and subcutaneous tissue, are a trend involving not only high costs for the health system but also and most important health problems in the subjects. Examples of these disorders are the obesity with the associated visceral fat accumulation, atherosclerosis and the fatty liver diseases.

In short, obesity is a condition in which excess body fat has accumulated to such an extent that it may have a negative effect on health.

Atherosclerosis is a pattern of the disease arteriosclerosis in which the wall of the artery develops abnormalities, called lesions. These lesions may lead to narrowing due to the build-up of atheromatous plaque. When severe, it can result in coronary artery disease, stroke, peripheral artery disease, or kidney problems, depending on which arteries are affected.

Fatty liver diseases (FLD), also known generically as hepatic steatosis (HS), is a condition where excess fat builds up in the liver. Fatty liver disease is typified as non-alcoholic fatty liver disease (NAFLD) and alcoholic liver disease. Westernized changes in diet and lifestyle can lead to the progression of some key risk factors, such as obesity, leptin, or insulin resistance (IR), dyslipidaemia and metabolic syndrome, which are associated with the development of NAFLD. NAFLD is one of the human diseases with more prevalence nowadays affecting almost one-third of the world's population. This disease is characterized by an excessive accumulation of fat in the liver and a low-grade inflammation. This abnormal lipid metabolism could affect lipid oxidation disturbing redox homeostasis in more advanced stages. Thus, accumulation of reactive species of oxygen (ROS) and consequently the increase of lipid peroxidation could induce hepatic cellular damage and fibrosis. NAFLD is classified in different grades according to severity and capacity to recover its healthy state: Non-alcoholic fatty liver (NAFL) with a simple steatosis; non-alcoholic steatohepatitis (NASH) with an advanced steatosis and inflammation. Moreover, NASH could end up developing fibrosis in advanced stages and finally progressing to irreversible stages like cirrhosis or hepatocarcinoma. Treatment of NAFLD is generally carried out by dietary changes and promotion of exercise to reduce body weight, unless a severe evolution makes compulsory a liver transplantation.

Cocktails of certain compounds have been tested in vivo as supplements in diet to boost the oxidation of fat and the synthesis of glutathione (GSH). GSH is required for preventing the accumulation of incomplete products of fatty acids oxidation. An example is the document of Mardinoglu et al., “Personal model-assisted identification of NAD+ and glutathione metabolism as intervention target in NAFLD”, Mol Syst Biol-2017, vol. no. 13:916. These authors tested in mice, fed with Western diet, including high levels of fat and sucrose, a cocktail consisting of serine, NAC (N-acetyl-L-cysteine), and nicotinamide riboside (NR). Serine was included into the cocktail since it can be easily converted to glycine whereas NAC was included since cysteine may be the limiting metabolite after the repletion of the glycine in the synthesis of GSH. The aim of this assay was to provide a proof-of-concept of a possible therapeutic strategy based on promoting the synthesis of GSH and NAD+ and resulting from an analysis by personalized genome-scale metabolic modelling of a human cohort with several degrees of hepatic steatosis. The rationale behind was to reduce the oxidative stress in lipid metabolism. From the GEMs analysis of the human cohort, the authors also concluded that the plasma levels of serine and glycine negatively correlated with HS, and that no significant correlation was present between HS and the plasma levels of cysteine and glutamine.

Another document proposing the supplementation of diet for mitigating high fat diet induced hepatic steatosis is the one of Mong et al., “Histidine and carnosine alleviated hepatic steatosis in mice consumed high saturated fat diet”, European Journal of Pharmacology 653 (2011) 82-88. The authors propose using histidine and the dipeptide carnosine to reduce hepatic steatosis after having observed that mice fed with these supplements lowered their body weight, the epididymal fat, and hepatic triglyceride and cholesterol levels, at the same time an improved insulin sensitivity and attenuated hyper-insulinemia was observed.

Among others, some of the roles attributed to histidine are those of being an anti-inflammatory and antioxidant, thus acting as a protective agent against liver. Firstly, this anti-inflammatory property of histidine supplementation has been related to a regulation of NF-kB and PPARα-induced pathways decreasing plasma concentrations of TNFα, IL6 and C-reactive protein and hepatic levels as well. Moreover, histidine has an anti-glycation action related to reduced inflammasome activation. In the other hand, the antioxidant characteristic of histidine treatment is due to that this amino acid is a free radical scavenger and it has the capacity to bind divalent metal ions.

Based on this later capacity as a free radical scavenger, some authors propose using a newly synthesized dimeric histidine (H-bihistidine) to treat non-alcoholic liver injury (NLI) and other free-radical-induced diseases (see. Zhao, Z.; Fu, C.; Zhang, Y.; Fu, A. Dimeric Histidine as a Novel Free Radical Scavenger Alleviates Non-Alcoholic Liver Injury. Antioxidants 2021, 10, 1529. https://doi.org/10.3390/antiox10101529). Although this is a good approach, the complexity of using a synthetic compound is always a drawback.

There is, thus, still a need of alternative approaches to face all these diseases or disorders associated with fat deposition in tissues.

### SUMMARY OF INVENTION

Inventors surprisingly found that a particular combination of three or more of histidine, cysteine, serine and carnosine were able to reduce liver injury and liver features of NAFLD, said injury seen macroscopically in terms of liver weight, whitish and fatty liver; and at biochemical level by the analysis of the levels of alanine aminotransferanse (ALT) and aspartate aminotransferase (AST). The combination of the compounds also decreased hepatic lipid content and liver steatosis. The supplementation of a diet with the combination of the compounds reduced hepatic inflammation associated to NAFLD, increased the antioxidant defence and antitumoral activity, and reduced resistance to insulin. In short, the combination of three or more of these compounds allowed reverting the NAFLD symptomatology in an animal model of the disease.

In addition, the inventors have found that the combination of compounds also allows to counteract the fat mass accretion associated to menopause in ovariectomized (OVX) rats and to decrease the loss of lean mass of OVX rats, contributing to a healthier profile of body composition. These compounds also ameliorate insulin resistance in this animal model. Thus, the authors have found that the combination of compounds contribute to revert metabolic changes associated to menopause in ovariectomized (OVX) rats.

Without being bound to any theory, the inventors propose that the combination of these compounds as ingredients in nutraceutical compositions, food supplements or as active ingredients in pharmaceutic compositions finally lead to an increase in the plasma histidine levels, which ultimately and due to a complexity of factors allow decreasing fat accumulation. This supposes a new approach in relation to the proposed used of histidine as radical scavenger or as anti-inflammatory in those disease involving fat accumulation, as previously disclosed. The approach is surprising since, moreover, histidine was only proposed as having a moderate effect on all these diseases, being the treatments in the prior art focused more on the promotion of the oxidation of fat and the synthesis of glutathione (GSH). Moreover, the proposed approach, resulting from the genuine observations made by the inventors, allow to safely face these disorders related with fat accumulation in several body tissue in subjects in which the control of the oxidative capacity of the fat is not jeopardized but that due to other mechanisms, that ultimately reduce histidine circulating levels, do also suffer from the clinical symptomatology.

This rationale could also be behind the reduction of visceral fat in obese subjects and in a reduction of LDL-cholesterol in a model of atherosclerosis.

Thus, a first aspect of the invention is a pharmaceutical or dietary supplement composition comprising, together with one or more pharmaceutically or nutraceutical acceptable excipients or carriers, a therapeutically or nutraceutical effective amount of at least three of histidine or a pharmaceutically or nutraceutical acceptable salt thereof or a solvate of any of both; cysteine or a pharmaceutically or nutraceutical acceptable salt thereof or a solvate of any of both, serine or a pharmaceutically or nutraceutical acceptable salt thereof or a solvate of any of both, and carnosine or a pharmaceutically or nutraceutical acceptable salt thereof or a solvate of any of both; and/or a therapeutically or nutraceutical effective amount of a peptide, said peptide comprising a sequence of amino acids including at least three of the histidine, cysteine, serine and carnosine, particularly, the pharmaceutical or dietary supplement composition comprises, together with one or more pharmaceutically or nutraceutical acceptable excipients or carriers: (i) a therapeutically or nutraceutical effective amount of cysteine or a pharmaceutically or nutraceutical acceptable salt thereof or a solvate of any of both, and a therapeutically or nutraceutical effective amount of at least two of: histidine or a pharmaceutically or nutraceutical acceptable salt thereof or a solvate of any of both, serine or a pharmaceutically or nutraceutical acceptable salt thereof or a solvate of any of both, and carnosine or a pharmaceutically or nutraceutical acceptable salt thereof or a solvate of any of both; and/or a therapeutically or nutraceutical effective amount of a peptide, said peptide comprising a sequence of amino acids including cysteine and at least two of the histidine, serine and carnosine; or, alternatively, (ii) a therapeutically or nutraceutical effective amount of N-acetylcysteine (NAC) or a pharmaceutically or nutraceutical acceptable salt thereof or a solvate of any of both, and a therapeutically or nutraceutical effective amount of at least two of: histidine or a pharmaceutically or nutraceutical acceptable salt thereof or a solvate of any of both, serine or a pharmaceutically or nutraceutical acceptable salt thereof or a solvate of any of both, and carnosine or a pharmaceutically or nutraceutical acceptable salt thereof or a solvate of any of both; and/or a therapeutically or nutraceutical effective amount of a peptide, said peptide comprising a sequence of amino acids including NAC and at least two of the histidine, serine and carnosine.

The pharmaceutical or dietary supplement composition can be used as ingredient in food products of common use, such as in beverages (i.e., sodas), dairy products (i.e., yogurts), bakery, etc., together with the common ingredients of these kind of foods. Thus, a second aspect of the invention is a food product in solid or liquid form comprising the pharmaceutical or dietary supplement composition as defined in the first aspect.

As will be illustrated in the examples, and as previously indicated, the composition or food comprising at least three of the listed compounds provided several beneficial effects in an animal model of NAFLD and in a menopause animal model Thus, another aspect of the invention is the composition as defined in the first aspect, or the food product of the second aspect, for use in therapy (i.e., as a medicament).

### BRIEF DESCRIPTION OF DRAWINGS

FIG. 1 shows the effects of treatments on (A) liver weight, (B) a representative macroscopic appearance of livers; and the (C) serum ALT (alanine amino-transferase); and (D) serum AST (aspartate amino-transferase). Data are mean±SEM; n=16 animals/group. *p<0.05, **p<0.01, ***p<0.001, ****p<0.0001 vs. control mice; #p<0.05, ##p<0.01, ###p<0.001 vs. HFHFr mice.

FIG. 2 shows the effects of treatments on (A) total liver lipid content; (B) total hepatic triglyceride (TG) content; (C) total hepatic cholesterol content. Data are mean±SEM; n=16 animals/group. **p<0.01, ***p<0.001, ****p<0.0001 vs. control mice; #p<0.05; #####p<0.0001 vs. HFHFr mice.

FIG. 3 shows liver histopathology and image analysis determined by (A) H&E (hematoxylin-eosin staining) and an amplification of the selected area showing a magnified area in the lower panel; (B) lipid droplets count; (C) lipid droplet surface field; and (D) NAFLD/NASH scoring table. st., steatosis. Bar=100 μm. Data are mean±SEM; n=6 animals/group. **p<0.01, ****p<0.0001 vs. control mice (C); ###p<0.001; ####p<0.0001 vs. HFHFr mice. AA mice are treated mice.

FIG. 4 shows the effects of treatments on liver insulin resistance. (A) A representative Western blot analysis with Akt activation (pAktS473), total Akt protein levels (T-Akt), housekeeping β-actin levels and protein loading with Ponceau-S membrane staining; (B) densitometry analysis of phosphorylated and total Akt ratio. Data are mean±SEM. n=5-6 animals/group. *p<0.05 vs. control mice. (C) Analysis of hepatic expression of key genes after the quaternary treatment of fatty acid beta-oxidation (carnitine palmitoyltransferase, Cpt1a), inflammation (chemokine (C-C motif) ligand 2, CCL2), lipolysis (hormone-sensitive, HSL; and lipin, Plin) and lipogenesis (fatty acid synthase, FASN). Data are expressed as mean±SEM; n=10-12 per condition. * p<0.05.

FIG. 5 shows the effects of the treatments on the levels of circulating cholesterol. In (A) there are depicted the levels of circulating (HDL), in (B) low-density lipoproteins (LDL) and in (C) the ratio LDL/HDL. Data are mean±SEM. n=16 animals/group. *p<0.05 **p<0.01, ***p<0.001, ****p<0.0001.

FIG. 6 shows the effects of the treatments on the body weight evolution after the treatment (A) and the weight of the main visceral depots after the treatment (B); epididymal white adipose tissue (EWAT); retroperitoneal white adipose tissue (RWAT) and mesenteric white adipose tissue (MWAT). (C) Growth curve. Circles represent “lean”, squares “obese”, and triangles “obese+4AA”. (D) Visceral adipose tissue histopathology and image analysis determined by H&E (hematoxylin-eosin staining). (E) BAT weight. (F) BAT histopathology and image analysis. (G) Gene expression analysis of the indicated genes in the BAT. (H) A representative Western blot analysis of the indicated proteins in the BAT and quantifications. (I) Thermography analysis of mice and quantifications. In (D) to (I), left columns represent “lean”, middle columns “obese”, and right columns “obese+4AA”. Data are mean±SEM. n=16 animals/group. *p<0.05 **p<0.01, ****p<0.0001.

FIG. 7 shows the effects of the treatment with the indicated amino acids alone or in combination on the Cpt1a, Ccl2, Hsl, Plin, and Fasn mRNA expression in human hepatocytes (HepG2) for 24 h with the following concentrations: Histidine 10 mM, carnosine 5 mM, cysteine 5 mM and serine 5 mM. These concentrations were conserved with the different combinations. Data are expressed as mean±SEM; n=3-4 per condition. *p<0.05; **p<0.01, ***p<0.001 vs. vehicle.

FIG. 8 shows effects of ovariectomy, 17B-E2 injections and the quaternary histidine+cysteine+serine+carnosine (4 AA) treatment on the evolution of body weight (A), fat mass (B) and lean mass (C) in sham-operated (SH) and ovariectomized (OVX) female Sprague-Dawley rats after 57 days of treatment. Body weight was recorded every 7 days, whereas fat and lean masses were documented every 28 days. Body weight gain in grams (D) was calculated as quotient of the final weight accumulated weekly throughout the study. Fat mass and lean mass gain were calculated as the difference in percentage between fat mass at the end of the experiment and the baseline point. Data are given as the mean±SEM (n=10). In each panel, different superscript lowercase letters (a,b,c,d) indicate significant different mean values among groups (one-way ANOVA and Duncan's post-hoc test, Welch test and Games-Howell post-hoc test or Kruskal-Wallis test and Mann-Whitney U post-hoc test, p<0.05). I: the effect of intervention, t: the effect of time, Ixt: the interaction between intervention type and time (RM-ANOVA, p<0.05). SH: Sham-operated rats, OVX: Ovariectomized rats, OVX-E2: OVX rats treated with 17β-oestradiol, OVX-4AA: OVX rats supplemented with the quaternary histidine+cysteine+serine+carnosine (4 AA). In (A) to (C), circles represent “SH”, triangles “OVX”, diamonds “OVX-E2”, and squares “OVX-4AA”.

FIG. 9 shows the effects of ovariectomy, 17B-E2 injections and the quaternary histidine+cysteine+serine+carnosine (4 AA) treatment on RWAT (A), MWAT (B), IWAT (C) depot weights, adiposity index (D), and circulating levels of leptin (E) in sham-operated (SH) and ovariectomized (OVX) rats after 57 days of intervention. Data are given as the mean±SEM (n=9-10). The adiposity index was computed as the sum of the IWAT, MWAT and RWAT depot weights (in grams) and is expressed as a percentage of body weight. In each figure, different superscript lowercase letters (a,b,c) indicate significant different mean values among groups (one-way ANOVA and Duncan's post-hoc test, p<0.05). I: effect of intervention. SH: Sham-operated rats, OVX: Ovariectomized rats, OVX-E2: OVX rats treated with 17β-oestradiol, OVX-4AA: OVX rats supplemented with the quaternary histidine related aminoacids (4AA). MWAT: mesenteric white adipose tissue; RWAT: retroperitoneal white adipose tissue; IWAT: inguinal white adipose tissue.

FIG. 10 shows plasma levels of insulin (a), glucose (b) and HOMA-IR (c) at the end of the study in sham-operated (SH) and ovariectomized (OVX) rats after 57 days of intervention. Data are given as the mean±SEM (n=9-10). The superscript lowercase letters (a,b,c) indicate significant different mean values among groups (one-way ANOVA and Duncan's post-hoc test, p<0.05). I: effect of intervention. SH: Sham-operated rats, OVX: Ovariectomized rats, OVX-E2: OVX rats treated with 17β-oestradiol, OVX-4AA: OVX rats supplemented with the quaternary histidine+cysteine+serine+carnosine (4 AA). HOMA-IR: homeostasis model assessment-estimated insulin resistance.

FIG. 11 shows associations of plasma histidine and steatosis degree with the gut microbiota and Hut genes in humans. a) Volcano plot of differential bacteria families associated with the circulating histidine levels in the discovery cohort (n=73) identified using the Analysis of Microbiomes with Bias Correction (ANCOM-BC) controlling for age, BMI, sex, and country. b) Volcano plot of differential bacteria families associated with the hepatic steatosis degree (liver biopsy) in the discovery cohort (n=73) identified using the Analysis of Microbiomes with Bias Correction (ANCOM-BC) controlling for age, BMI, sex, and country. The log 2 (Fold Change) associated with a unit change in the plasma histidine levels and the −log 10 (p-values) adjusted for multiple testing are plotted for each taxon. Significantly different taxa are coloured according to phylum. c) Histidine utilisation pathways. The first three pathways appear to be universal. There are two different degradation pathways for formiminoglutamate depending on the genera: hydrolyzation to formamide and glutamate or hydrolyzation to formylglutamate and subsequent hydrolyzation to formate and glutamate. d) Violin plots of the centered log ratio-transformed microbial genes involved in histidine utilisation hutH, hutU, hutI and hutG, respectively, in subjects with steatosis degree lower or higher than 33%. e) qPCR of microbial genes expression involved in histidine utilization (hutH and hutG). Data are mean±SEM. *p<0.05, **p<0.01, ***p<0.001, ****p<0.0001 vs. NAFLD mice

### DETAILED DESCRIPTION OF THE INVENTION

All terms as used herein in this application, unless otherwise stated, shall be understood in their ordinary meaning as known in the art. Other more specific definitions for certain terms as used in the present application are as set forth below and are intended to apply uniformly through-out the specification and claims unless an otherwise expressly set out definition provides a broader definition.

As used herein, the indefinite articles “a” and “an” are synonymous with “at least one” or “one or more.” Unless indicated otherwise, definite articles used herein, such as “the” also include the plural of the noun.

For the purposes of the present invention, any ranges given include both the lower and the upper end-points of the range.

As used herein, the term “pharmaceutical acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutical acceptable salts are well known in the art. Examples of pharmaceutical acceptable, nontoxic acid addition salts are salts of an amino group formed with inorganic acids such as hydrochloric acid, hydrobromic acid, phosphoric acid, sulfuric acid and perchloric acid or with organic acids such as acetic acid, trifluoroacetic acid, oxalic acid, maleic acid, tartaric acid, citric acid, succinic acid or malonic acid or by using other methods used in the art such as ion exchange. Other pharmaceutical acceptable salts include adipate, alginate, ascorbate, aspartate, benzenesulfonate, benzoate, bisulphate, borate, butyrate, camphorate, camphorsulfonate, citrate, cyclopentanepropionate, digluconate, dodecylsulphate, ethanesulfonate, formate, fumarate, glucoheptonate, glycerophosphate, gluconate, hemisulphate, heptanoate, hexanoate, hydroiodide, 2-hydroxy-ethanesulfonate, lactobionate, lactate, laurate, lauryl sulphate, malate, maleate, malonate, methanesulfonate, 2-naphthalenesulfonate, nicotinate, nitrate, oleate, oxalate, palmitate, pamoate, pectinate, persulphate, 3-phenylpropionate, phosphate, picrate, pivalate, propionate, stearate, succinate, sulphate, tartrate, thiocyanate, p-toluenesulfonate, undecanoate, valerate salts, and the like. Salts derived from appropriate bases include alkali metal, alkaline earth metal, and ammonium. Representative alkali or alkaline earth metal salts include sodium, lithium, potassium, calcium, magnesium, and the like. Further pharmaceutical acceptable salts include, when appropriate, nontoxic ammonium, quaternary ammonium, and amine cations formed using counterions such as halide, hydroxide, carboxylate, sulphate, phosphate, nitrate, lower alkyl sulfonate and aryl sulfonate.

The term “solvate” refers to a molecular complex comprising the compound of interest (i.e., of L-histidine, L-cysteine, L-serine and L-carnosine or a pharmaceutically acceptable salt thereof), and a stoichiometric or non-stoichiometric amount of one or more solvent molecules (e.g., water) bound by non-covalent intermolecular forces. When the one or more solvent molecules forming part of the molecular complex is water, the solvate is a hydrate. Methods of solvation are generally known within the art.

The term “pharmaceutical” encompasses also the concept of “veterinary composition”. Thus, they relate to compositions that are therapeutically effective when administered by any desired or applicable route to any animal, including humans.

The expression “therapeutically effective amount” as used herein, refers to the amount of a compound that, when administered, is sufficient to prevent development of, or alleviate to some extent, one or more of the symptoms of the disease which is addressed. The particular dose of compound administered according to this invention will of course be determined by the particular circumstances surrounding the case, including the compound administered, the route of administration, the particular condition being treated, and the similar considerations.

The expression “pharmaceutically or nutraceutical acceptable excipients or carriers” refers to pharmaceutically acceptable materials, compositions, or vehicles. Each component must be pharmaceutically acceptable in the sense of being compatible with the other ingredients of the pharmaceutical composition. It must also be suitable for use in contact with the tissue or organ of humans and animals without excessive toxicity, irritation, allergic response, immunogenicity or other problems or complications commensurate with a reasonable benefit/risk ratio. Examples of suitable acceptable excipients are solvents, dispersion media, diluents, or other liquid vehicles, dispersion or suspension aids, surface active agents, isotonic agents, thickening or emulsifying agents, preservatives, solid binders, lubricants and the like. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this invention.

The expression “nutraceutical effective amount” as used herein, refers to the amount of a compound that, when administered, is sufficient to provide a physiological benefit to the subject in certain particular metabolic aspects. The particular dose of compound administered according to this invention will of course be determined by the particular circumstances surrounding the case, including the compound administered, the route of administration, the particular condition being treated, and the similar considerations.

A “dietary supplement composition”, also called herewith as “nutraceutical composition” or “food supplement” is a composition providing a physiological benefit to the subject in certain particular metabolic aspects. Although they cannot be labelled with drug claims, they can bear health claims and nutrition claims, according to the regulatory of health authorities. Under this category several compositions are included, which differ depending on the regulatory of the countries. In general, it is a product taken by mouth that contains a ‘dietary ingredient’ intended to supplement the diet. The ‘dietary ingredients’ in these products may include: vitamins, minerals, herbs or other botanicals, amino acids, and substances such as enzymes, organ tissues, glandular, and metabolites. Dietary supplements can also be extracts or concentrates and may be found in many forms such as tablets, capsules, softgels, gelcaps, liquids, or powders.

“Functional foods”, also called in this description as “food products” are fortified or enriched during processing and then marketed as providing some benefit to consumers.

The term “NAC” or “N-acetylcysteine” or “acetylcysteine” as used herein, refers to the acetylated form of the amino acid L-cysteine and that has IUPAC name (2R)-2-acetamido-3-sulfanylpropanoic acid. As the skilled person would appreciate, NAC is well-stablished cysteine pro-drug, therefore cysteine and NAC can be used interchangeably in the compositions of the invention.

As previously indicated, a first aspect of the invention is a pharmaceutical or dietary supplement composition comprising, together with one or more pharmaceutically or nutraceutical acceptable excipients or carriers, a therapeutically or nutraceutical effective amount of at least three of histidine or a pharmaceutically or nutraceutical acceptable salt thereof or a solvate of any of both; cysteine or a pharmaceutically or nutraceutical acceptable salt thereof or a solvate of any of both, serine or a pharmaceutically or nutraceutical acceptable salt thereof or a solvate of any of both, and carnosine or a pharmaceutically or nutraceutical acceptable salt thereof or a solvate of any of both; and/or a therapeutically or nutraceutical effective amount of a peptide, said peptide comprising a sequence of amino acids including at least three selected of the group of compounds consisting of the histidine, cysteine, serine and carnosine, particularly, the pharmaceutical or dietary supplement composition comprises, together with one or more pharmaceutically or nutraceutical acceptable excipients or carriers:
(i) a therapeutically or nutraceutical effective amount of cysteine or a pharmaceutically or nutraceutical acceptable salt thereof or a solvate of any of both, and a therapeutically or nutraceutical effective amount of at least two of: histidine or a pharmaceutically or nutraceutical acceptable salt thereof or a solvate of any of both, serine or a pharmaceutically or nutraceutical acceptable salt thereof or a solvate of any of both, and carnosine or a pharmaceutically or nutraceutical acceptable salt thereof or a solvate of any of both; and/or a therapeutically or nutraceutical effective amount of a peptide, said peptide comprising a sequence of amino acids including cysteine and at least two of the histidine, serine and carnosine; or, alternatively,
(ii) a therapeutically or nutraceutical effective amount of N-acetylcysteine (NAC) or a pharmaceutically or nutraceutical acceptable salt thereof or a solvate of any of both, and a therapeutically or nutraceutical effective amount of at least two of: histidine or a pharmaceutically or nutraceutical acceptable salt thereof or a solvate of any of both, serine or a pharmaceutically or nutraceutical acceptable salt thereof or a solvate of any of both, and carnosine or a pharmaceutically or nutraceutical acceptable salt thereof or a solvate of any of both; and/or a therapeutically or nutraceutical effective amount of a peptide, said peptide comprising a sequence of amino acids including NAC and at least two of the histidine, serine and carnosine.

In a particular embodiment, the pharmaceutical composition or dietary supplement composition comprises together with one or more pharmaceutically or nutraceutical acceptable excipients or carriers, a therapeutically or nutraceutical effective amount of at least three of histidine or a pharmaceutically or nutraceutical acceptable salt thereof or a solvate of any of both; cysteine or a pharmaceutically or nutraceutical acceptable salt thereof or a solvate of any of both, serine or a pharmaceutically or nutraceutical acceptable salt thereof or a solvate of any of both, and carnosine or a pharmaceutically or nutraceutical acceptable salt thereof or a solvate of any of both.

In a particular embodiment, the pharmaceutical or dietary supplement composition comprises, together with one or more pharmaceutically or nutraceutical acceptable excipients or carriers:
(i) a therapeutically or nutraceutical effective amount of cysteine or a pharmaceutically or nutraceutical acceptable salt thereof or a solvate of any of both, and a therapeutically or nutraceutical effective amount of at least two of: histidine or a pharmaceutically or nutraceutical acceptable salt thereof or a solvate of any of both, serine or a pharmaceutically or nutraceutical acceptable salt thereof or a solvate of any of both, and carnosine or a pharmaceutically or nutraceutical acceptable salt thereof or a solvate of any of both; and/or a therapeutically or nutraceutical effective amount of a peptide, said peptide comprising a sequence of amino acids including cysteine and at least two of the histidine, serine and carnosine; or, alternatively,
(ii) a therapeutically or nutraceutical effective amount of N-acetylcysteine (NAC) or a pharmaceutically or nutraceutical acceptable salt thereof or a solvate of any of both, and a therapeutically or nutraceutical effective amount of at least two of: histidine or a pharmaceutically or nutraceutical acceptable salt thereof or a solvate of any of both, serine or a pharmaceutically or nutraceutical acceptable salt thereof or a solvate of any of both, and carnosine or a pharmaceutically or nutraceutical acceptable salt thereof or a solvate of any of both; and/or a therapeutically or nutraceutical effective amount of a peptide, said peptide comprising a sequence of amino acids including NAC and at least two of the histidine, serine and carnosine.

In another particular embodiment, the pharmaceutical or dietary supplement composition comprises, together with one or more pharmaceutically or nutraceutical acceptable excipients or carriers:
(i) a therapeutically or nutraceutical effective amount of cysteine or a pharmaceutically or nutraceutical acceptable salt thereof or a solvate of any of both, and a therapeutically or nutraceutical effective amount of at least two of: histidine or a pharmaceutically or nutraceutical acceptable salt thereof or a solvate of any of both, serine or a pharmaceutically or nutraceutical acceptable salt thereof or a solvate of any of both, and carnosine or a pharmaceutically or nutraceutical acceptable salt thereof or a solvate of any of both; or, alternatively,
(ii) a therapeutically or nutraceutical effective amount of N-acetylcysteine (NAC) or a pharmaceutically or nutraceutical acceptable salt thereof or a solvate of any of both, and a therapeutically or nutraceutical effective amount of at least two of: histidine or a pharmaceutically or nutraceutical acceptable salt thereof or a solvate of any of both, serine or a pharmaceutically or nutraceutical acceptable salt thereof or a solvate of any of both, and carnosine or a pharmaceutically or nutraceutical acceptable salt thereof or a solvate of any of both.

In another particular embodiment, the pharmaceutical or dietary supplement composition comprises, together with one or more pharmaceutically or nutraceutical acceptable excipients or carriers:
(i) a therapeutically or nutraceutical effective amount of cysteine or a pharmaceutically or nutraceutical acceptable salt thereof or a solvate of any of both, a therapeutically or nutraceutical effective amount of histidine or a pharmaceutically or nutraceutical acceptable salt thereof or a solvate of any of both, a therapeutically or nutraceutical effective amount of serine or a pharmaceutically or nutraceutical acceptable salt thereof or a solvate of any of both and a therapeutically or nutraceutical effective amount carnosine or a pharmaceutically or nutraceutical acceptable salt thereof or a solvate of any of both; and/or a therapeutically or nutraceutical effective amount of a peptide, said peptide comprising a sequence of amino acids including cysteine, histidine, serine and carnosine; or, alternatively,
(ii) a therapeutically or nutraceutical effective amount of NAC or a pharmaceutically or nutraceutical acceptable salt thereof or a solvate of any of both, a therapeutically or nutraceutical effective amount of histidine or a pharmaceutically or nutraceutical acceptable salt thereof or a solvate of any of both, a therapeutically or nutraceutical effective amount of serine or a pharmaceutically or nutraceutical acceptable salt thereof or a solvate of any of both, and a therapeutically or nutraceutical effective amount carnosine or a pharmaceutically or nutraceutical acceptable salt thereof or a solvate of any of both; and/or a therapeutically or nutraceutical effective amount of a peptide, said peptide comprising a sequence of amino acids including NAC, histidine, serine and carnosine.

In another particular embodiment, the pharmaceutical or dietary supplement composition comprises, together with one or more pharmaceutically or nutraceutical acceptable excipients or carriers:
(i) a therapeutically or nutraceutical effective amount of cysteine or a pharmaceutically or nutraceutical acceptable salt thereof or a solvate of any of both, a therapeutically or nutraceutical effective amount of histidine or a pharmaceutically or nutraceutical acceptable salt thereof or a solvate of any of both, a therapeutically or nutraceutical effective amount of serine or a pharmaceutically or nutraceutical acceptable salt thereof or a solvate of any of both, and a therapeutically or nutraceutical effective amount carnosine or a pharmaceutically or nutraceutical acceptable salt thereof or a solvate of any of both; or, alternatively,
(ii) a therapeutically or nutraceutical effective amount of NAC or a pharmaceutically or nutraceutical acceptable salt thereof or a solvate of any of both, a therapeutically or nutraceutical effective amount of histidine or a pharmaceutically or nutraceutical acceptable salt thereof or a solvate of any of both, a therapeutically or nutraceutical effective amount of serine or a pharmaceutically or nutraceutical acceptable salt thereof or a solvate of any of both, and a therapeutically or nutraceutical effective amount carnosine or a pharmaceutically or nutraceutical acceptable salt thereof or a solvate of any of both.

In a more particular embodiment, the pharmaceutical or dietary supplement composition comprises, together with one or more pharmaceutically or nutraceutical acceptable excipients or carriers, a therapeutically or nutraceutical effective amount of N-acetylcysteine (NAC) or a pharmaceutically or nutraceutical acceptable salt thereof or a solvate of any of both, and a therapeutically or nutraceutical effective amount of at least two of: histidine or a pharmaceutically or nutraceutical acceptable salt thereof or a solvate of any of both, serine or a pharmaceutically or nutraceutical acceptable salt thereof or a solvate of any of both, and carnosine or a pharmaceutically or nutraceutical acceptable salt thereof or a solvate of any of both.

In a more particular embodiment, the pharmaceutical or dietary supplement composition comprises, together with one or more pharmaceutically or nutraceutical acceptable excipients or carriers, a therapeutically or nutraceutical effective amount of NAC or a pharmaceutically or nutraceutical acceptable salt thereof or a solvate of any of both, a therapeutically or nutraceutical effective amount of histidine or a pharmaceutically or nutraceutical acceptable salt thereof or a solvate of any of both, a therapeutically or nutraceutical effective amount of serine or a pharmaceutically or nutraceutical acceptable salt thereof or a solvate of any of both, and a therapeutically or nutraceutical effective amount carnosine or a pharmaceutically or nutraceutical acceptable salt thereof or a solvate of any of both.

In a particular embodiment, the pharmaceutical composition or dietary supplement composition is that wherein the therapeutically effective amount of cysteine or a pharmaceutically or nutraceutical acceptable salt thereof or a solvate of any of both is that giving a dose from 2 to 550 mg per Kg of the subject; the therapeutically or nutraceutical effective amount of histidine or a pharmaceutically or nutraceutical acceptable salt thereof or a solvate of any of both is that giving a dose from 7.5 mg to 250 mg per Kg of the subject; the therapeutically effective amount of serine or a pharmaceutically or nutraceutical acceptable salt thereof or a solvate of any of both is that giving a dose from 1.3 to 250 mg per Kg of the subject; and the therapeutically effective amount of carnosine or a pharmaceutically or nutraceutical acceptable salt thereof or a solvate of any of both is that giving a dose from 4.5 to 200 mg per Kg of the subject.

In a particular embodiment, the pharmaceutical composition or dietary supplement composition is that wherein the therapeutically or nutraceutical effective amount of histidine or a pharmaceutically or nutraceutical acceptable salt thereof or a solvate of any of both is that giving a dose from 10 mg to 250 mg per Kg of the subject; the therapeutically or nutraceutical effective amount of cysteine or a pharmaceutically or nutraceutical acceptable salt thereof or a solvate of any of both is that giving a dose from 30 to 550 mg per Kg of the subject; the therapeutically or nutraceutical effective amount of serine or a pharmaceutically or nutraceutical acceptable salt thereof or a solvate of any of both is that giving a dose from 10 to 250 mg per Kg of the subject; and the therapeutically or nutraceutical effective amount of carnosine or a pharmaceutically or nutraceutical acceptable salt thereof or a solvate of any of both is that giving a dose from 10 to 200 mg per Kg of the subject.

In another particular embodiment, the pharmaceutical composition or dietary supplement composition is that wherein the therapeutically effective amount of NAC or a pharmaceutically or nutraceutical acceptable salt thereof or a solvate of any of both is that giving a dose from 2 to 550 mg per Kg of the subject; the therapeutically or nutraceutical effective amount of histidine or a pharmaceutically or nutraceutical acceptable salt thereof or a solvate of any of both is that giving a dose from 7.5 mg to 250 mg per Kg of the subject; the therapeutically effective amount of serine or a pharmaceutically or nutraceutical acceptable salt thereof or a solvate of any of both is that giving a dose from 1.3 to 250 mg per Kg of the subject; and the therapeutically effective amount of carnosine or a pharmaceutically or nutraceutical acceptable salt thereof or a solvate of any of both is that giving a dose from 4.5 to 200 mg per Kg of the subject.

In another particular embodiment, the pharmaceutical composition or dietary supplement composition is that wherein the therapeutically effective amount of NAC or a pharmaceutically or nutraceutical acceptable salt thereof or a solvate of any of both is that giving a dose from 30 to 550 mg per Kg of the subject; the therapeutically or nutraceutical effective amount of histidine or a pharmaceutically or nutraceutical acceptable salt thereof or a solvate of any of both is that giving a dose from 10 mg to 250 mg per Kg of the subject; the therapeutically effective amount of serine or a pharmaceutically or nutraceutical acceptable salt thereof or a solvate of any of both is that giving a dose from 10 to 250 mg per Kg of the subject; and the therapeutically effective amount of carnosine or a pharmaceutically or nutraceutical acceptable salt thereof or a solvate of any of both is that giving a dose from 10 to 200 mg per Kg of the subject.

The indicated therapeutically or nutraceutical effective amounts are those referred to the weight of an animal, in particular a mammalian, including mice and human. Indeed, the amounts and doses are commonly determined by the skilled person in the art by means of the human equivalent dose (HED) calculations the skilled person can find the values for human (Guidance for Industry Estimating the Maximum Safe Starting Dose in Initial Clinical Trials for Therapeutics in Adult Healthy Volunteers U.S. Department of Health and Human Services Food and Drug Administration Center for Drug Evaluation and Research (CDER) July 2005 Pharmacology and Toxicology).

For example, a dose in mice of 210 mg/Kg of histidine, serine and carnosine supposes an HED of 17.027 mg/Kg (median human weight of 70 kg). In the same way, a dose of 490 mg/Kg supposes an HED of 39.730 mg/Kg. The amount per Kg of subject is, in a particular embodiment of the invention the one used to calculate the dose (i.e., amount) per day. Particular daily doses in human are from 660 mg/day to 1500 mg/day for histidine, serine and carnosine (or any salt or solvate thereof), and from 1700 mg/day to 3000 mg/day for cysteine (or any salt or solvate thereof).

In a particular embodiment, the therapeutically or nutraceutical effective amount of any one of histidine, serine and carnosine or a pharmaceutically acceptable salt or solvate thereof is that giving a dose from 12 to 230 mg/Kg, or even more in particular from 17 to 210 mg/Kg.

In a particular embodiment, the therapeutically or nutraceutical effective amount of histidine, serine and carnosine or a pharmaceutically acceptable salt or solvate thereof is that giving a dose from 12 to 25 mg/Kg in humans, or an amount selected from 0.5, 1, 1.5, 2, 2.5, 3, 3.5, 4, 4.5, 5, 5.5, 6, 6.5, 7, 7.5, 8, 9, 10, 11, 12, 13, 14, 15, 16, 17, 18, 19, 20, 21, 22, 23, 24 and 25 mg/Kg. In mice it is of 200 mg/Kg to 230 mg/Kg, and more in particular is 210 mg/Kg.

In a particular embodiment, the therapeutically or nutraceutical effective amount of histidine, serine and carnosine or a pharmaceutically acceptable salt or solvate thereof is that giving a dose of 0.5-25, 1-25, 1.3-25, 1.5-25, 2.5-25, 4-25, 5-25, 6-25, 7.5-25, 8-25, 9-25, 10-25, 12-25, 15-25, 17-25, or 20-25 mg/kg in humans, particularly 15-25 mg/kg in humans. In another particular embodiment, the therapeutically or nutraceutical effective amount of histidine, serine and carnosine or a pharmaceutically acceptable salt or solvate thereof is that giving a dose of 10-25, 12-22, 15-20, or 16-18 mg/kg in humans, particularly 15-20 mg/kg. In another more particular embodiment, optionally in combination with any of the embodiments above or below, the therapeutically or nutraceutical effective amount of cysteine or a pharmaceutically or nutraceutical acceptable salt or solvate thereof is that giving a dose from 35 mg/Kg to 500 mg/Kg. In another more particular embodiment, optionally in combination with any of the embodiments above or below, the therapeutically or nutraceutical effective amount of cysteine or a pharmaceutically or nutraceutical acceptable salt or solvate thereof is that giving a dose from 35 to 50 mg/Kg in humans, or an amount selected from 0.5, 1, 2, 3, 4, 5, 6, 7, 8, 9, 10, 15, 20, 25, 30, 35, 36, 37, 38, 39, 39.5, 40, 41, 42, 43, 44, 45, 46, 47, 48, 49 and 50 mg/Kg. In mice it is of 480 mg/Kg to 500 mg/Kg, and more in particular is 490 mg/Kg.

In another particular embodiment, optionally in combination with any of the embodiments above or below, the therapeutically or nutraceutical effective amount of cysteine or a pharmaceutically or nutraceutical acceptable salt or solvate thereof is that giving a dose of 1-10, 1.5-7.5, or 2-5 mg/kg in humans, particularly 2-5 mg/kg in humans.

In another particular embodiment, optionally in combination with any of the embodiments above or below, the therapeutically or nutraceutical effective amount of cysteine or a pharmaceutically or nutraceutical acceptable salt or solvate thereof is that giving a dose of 20-55, 25-50, 30-45, or 35-40, particularly 35-40 mg/kg in humans.

In another more particular embodiment, optionally in combination with any of the embodiments above or below, the therapeutically or nutraceutical effective amount of NAC or a pharmaceutically or nutraceutical acceptable salt or solvate thereof is that giving a dose from 35 mg/Kg to 500 mg/Kg. In another more particular embodiment, optionally in combination with any of the embodiments above or below, the therapeutically or nutraceutical effective amount of NAC or a pharmaceutically or nutraceutical acceptable salt or solvate thereof is that giving a dose from 35 to 50 mg/Kg in humans, or an amount selected from 0.5, 1, 2, 3, 4, 5, 6, 7, 8, 9, 10, 15, 20, 25, 30, 35, 36, 37, 38, 39, 39.5, 40, 41, 42, 43, 44, 45, 46, 47, 48, 49 and 50 mg/Kg. In mice it is of 480 mg/Kg to 500 mg/Kg, and more in particular is 490 mg/Kg.

In another particular embodiment, optionally in combination with any of the embodiments above or below, the therapeutically or nutraceutical effective amount of NAC or a pharmaceutically or nutraceutical acceptable salt or solvate thereof is that giving a dose of 1-10, 1.5-7.5, or 2-5 mg/kg in humans, particularly 2-5 mg/kg in humans.

In another particular embodiment, optionally in combination with any of the embodiments above or below, the therapeutically or nutraceutical effective amount of cysteine or a pharmaceutically or nutraceutical acceptable salt or solvate thereof is that giving a dose of 20-55, 25-50, 30-45, or 35-40, particularly 35-40 mg/kg in humans.

In a more particular embodiment, the pharmaceutical composition or dietary supplement composition is that wherein the therapeutically effective amount of cysteine or a pharmaceutically or nutraceutical acceptable salt thereof or a solvate of any of both is that giving a dose from 1 to 7.5 mg per Kg of the subject, wherein the subject is preferably a human; the therapeutically or nutraceutical effective amount of histidine or a pharmaceutically or nutraceutical acceptable salt thereof or a solvate of any of both is that giving a dose from 5 mg to 25 mg per Kg of the subject, wherein the subject is preferably a human; the therapeutically effective amount of serine or a pharmaceutically or nutraceutical acceptable salt thereof or a solvate of any of both is that giving a dose from 1 to 5 mg per Kg of the subject, wherein the subject is preferably a human; and the therapeutically effective amount of carnosine or a pharmaceutically or nutraceutical acceptable salt thereof or a solvate of any of both is that giving a dose from 2 to 15 mg per Kg of the subject, wherein the subject is preferably a human.

In an even more particular embodiment, the pharmaceutical composition or dietary supplement composition is that wherein the therapeutically effective amount of cysteine or a pharmaceutically or nutraceutical acceptable salt thereof or a solvate of any of both is that giving a dose from 2 to 5 mg per Kg of the subject, wherein the subject is preferably a human; the therapeutically or nutraceutical effective amount of histidine or a pharmaceutically or nutraceutical acceptable salt thereof or a solvate of any of both is that giving a dose from 7.5 to 20 mg per Kg of the subject, wherein the subject is preferably a human; the therapeutically effective amount of serine or a pharmaceutically or nutraceutical acceptable salt thereof or a solvate of any of both is that giving a dose from 1.3 to 3.3 mg per Kg of the subject, wherein the subject is preferably a human; and the therapeutically effective amount of carnosine or a pharmaceutically or nutraceutical acceptable salt thereof or a solvate of any of both is that giving a dose from 4.5 to 10 mg per Kg of the subject, wherein the subject is preferably a human.

In a more particular embodiment, the pharmaceutical composition or dietary supplement composition is that wherein the therapeutically effective amount of NAC or a pharmaceutically or nutraceutical acceptable salt thereof or a solvate of any of both is that giving a dose from 1 to 7.5 mg per Kg of the subject, wherein the subject is preferably a human; the therapeutically or nutraceutical effective amount of histidine or a pharmaceutically or nutraceutical acceptable salt thereof or a solvate of any of both is that giving a dose from 5 mg to 25 mg per Kg of the subject, wherein the subject is preferably a human; the therapeutically effective amount of serine or a pharmaceutically or nutraceutical acceptable salt thereof or a solvate of any of both is that giving a dose from 1 to 5 mg per Kg of the subject, wherein the subject is preferably a human; and the therapeutically effective amount of carnosine or a pharmaceutically or nutraceutical acceptable salt thereof or a solvate of any of both is that giving a dose from 2 to 15 mg per Kg of the subject, wherein the subject is preferably a human.

In an even more particular embodiment, the pharmaceutical composition or dietary supplement composition is that wherein the therapeutically effective amount of NAC or a pharmaceutically or nutraceutical acceptable salt thereof or a solvate of any of both is that giving a dose from 2 to 5 mg per Kg of the subject, wherein the subject is preferably a human; the therapeutically or nutraceutical effective amount of histidine or a pharmaceutically or nutraceutical acceptable salt thereof or a solvate of any of both is that giving a dose from 7.5 to 20 mg per Kg of the subject, wherein the subject is preferably a human; the therapeutically effective amount of serine or a pharmaceutically or nutraceutical acceptable salt thereof or a solvate of any of both is that giving a dose from 1.3 to 3.3 mg per Kg of the subject, wherein the subject is preferably a human; and the therapeutically effective amount of carnosine or a pharmaceutically or nutraceutical acceptable salt thereof or a solvate of any of both is that giving a dose from 4.5 to 10 mg per Kg of the subject, wherein the subject is preferably a human.

In a more particular embodiment, the pharmaceutical composition or dietary supplement composition is that wherein the therapeutically effective amount of cysteine or a pharmaceutically or nutraceutical acceptable salt thereof or a solvate of any of both is that giving a dose of 35-40 mg/kg of the subject, wherein the subject is preferably a human; the therapeutically or nutraceutical effective amount of histidine or a pharmaceutically or nutraceutical acceptable salt thereof or a solvate of any of both is that giving a dose of 15-20 mg/kg of the subject, wherein the subject is preferably a human; the therapeutically effective amount of serine or a pharmaceutically or nutraceutical acceptable salt thereof or a solvate of any of both is that giving a dose of 15-20 mg/kg of the subject, wherein the subject is preferably a human; and the therapeutically effective amount of carnosine or a pharmaceutically or nutraceutical acceptable salt thereof or a solvate of any of both is that giving a dose of 15-20 mg/kg of the subject, wherein the subject is preferably a human.

In a more particular embodiment, the pharmaceutical composition or dietary supplement composition is that wherein the therapeutically effective amount of NAC or a pharmaceutically or nutraceutical acceptable salt thereof or a solvate of any of both is that giving a dose of 35-40 mg/kg of the subject, wherein the subject is preferably a human; the therapeutically or nutraceutical effective amount of histidine or a pharmaceutically or nutraceutical acceptable salt thereof or a solvate of any of both is that giving a dose of 15-20 mg/kg of the subject, wherein the subject is preferably a human; the therapeutically effective amount of serine or a pharmaceutically or nutraceutical acceptable salt thereof or a solvate of any of both is that giving a dose of 15-20 mg/kg of the subject, wherein the subject is preferably a human; and the therapeutically effective amount of carnosine or a pharmaceutically or nutraceutical acceptable salt thereof or a solvate of any of both is that giving a dose of 15-20 mg/kg of the subject, wherein the subject is preferably a human.

In another particular embodiment of the first aspect, the composition comprises a therapeutically or nutraceutical effective amount of at least three of: L-histidine or a pharmaceutically or nutraceutical acceptable salt thereof or a solvate of any of both, L-cysteine or a pharmaceutically or nutraceutical acceptable salt thereof or a solvate of any of both, L-serine or a pharmaceutically or nutraceutical acceptable salt thereof or a solvate of any of both, and L-carnosine or a pharmaceutically or nutraceutical acceptable salt thereof or a solvate of any of both.

Indeed, the amino acids in the composition can be in any of the D or L enantiomers, although they are in a particular embodiment L-amino acids. In the same way the dipeptide carnosine can be in the form of L-carnosine or D-carnosine, although in a particular embodiment is L-carnosine. In an embodiment, the compositions of the invention include any combination of any of the compounds in L- or D-forms. In another embodiment, the compositions of the invention include any combination of any of the amino acids histidine, serine, carnosine and/or cysteine in L- or D-forms.

In a particular embodiment of the first aspect, the composition comprises:
(i) a therapeutically or nutraceutical effective amount of L-cysteine or a pharmaceutically or nutraceutical acceptable salt thereof or a solvate of any of both, and a therapeutically or nutraceutical effective amount of at least two of: L-histidine or a pharmaceutically or nutraceutical acceptable salt thereof or a solvate of any of both, L-serine or a pharmaceutically or nutraceutical acceptable salt thereof or a solvate of any of both, and L-carnosine or a pharmaceutically or nutraceutical acceptable salt thereof or a solvate of any of both; or, alternatively,
(ii) a therapeutically or nutraceutical effective amount of N-acetylcysteine (NAC) or a pharmaceutically or nutraceutical acceptable salt thereof or a solvate of any of both, and a therapeutically or nutraceutical effective amount of at least two of: L-histidine or a pharmaceutically or nutraceutical acceptable salt thereof or a solvate of any of both, L-serine or a pharmaceutically or nutraceutical acceptable salt thereof or a solvate of any of both, and L-carnosine or a pharmaceutically or nutraceutical acceptable salt thereof or a solvate of any of both.

In another particular embodiment of the first aspect, the composition comprises:
(i) a therapeutically or nutraceutical effective amount of L-cysteine or a pharmaceutically or nutraceutical acceptable salt thereof or a solvate of any of both, a therapeutically or nutraceutical effective amount of L-histidine or a pharmaceutically or nutraceutical acceptable salt thereof or a solvate of any of both, a therapeutically or nutraceutical effective amount of L-serine or a pharmaceutically or nutraceutical acceptable salt thereof or a solvate of any of both and a therapeutically or nutraceutical effective amount L-carnosine or a pharmaceutically or nutraceutical acceptable salt thereof or a solvate of any of both; or, alternatively,
(ii) a therapeutically or nutraceutical effective amount of NAC or a pharmaceutically or nutraceutical acceptable salt thereof or a solvate of any of both, a therapeutically or nutraceutical effective amount of L-histidine or a pharmaceutically or nutraceutical acceptable salt thereof or a solvate of any of both, a therapeutically or nutraceutical effective amount of L-serine or a pharmaceutically or nutraceutical acceptable salt thereof or a solvate of any of both, and a therapeutically or nutraceutical effective amount L-carnosine or a pharmaceutically or nutraceutical acceptable salt thereof or a solvate of any of both.

In another particular embodiment of the pharmaceutical composition according to the first aspect, the pharmaceutically or nutraceutical acceptable salt of histidine and of cysteine is the hydrochloride salt, more in particular the hydrochloride salt of L-histidine and of L-cysteine. Even more in particular, the pharmaceutically acceptable salt of L-histidine is L-histidine monohydrochloride monohydrate, and the pharmaceutically acceptable salt of L-cysteine is L-cysteine hydrochloride.

In another particular embodiment of the pharmaceutical composition according to the first aspect, the pharmaceutically or nutraceutical acceptable salt of histidine is the hydrochloride salt, more in particular the hydrochloride salt of L-histidine. Even more in particular, the pharmaceutically acceptable salt of L-histidine is L-histidine monohydrochloride monohydrate.

In another particular embodiment of the pharmaceutical composition according to the first aspect, the pharmaceutically or nutraceutical acceptable salt of cysteine is the hydrochloride salt, more in particular the hydrochloride salt of L-cysteine. Even more in particular, the pharmaceutically acceptable salt of L-cysteine is L-cysteine hydrochloride.

In a more particular embodiment of the pharmaceutical and dietary supplement of the first aspect, they comprise at least three of the indicated ingredients being at least one of them histidine, more in particular L-histidine or a salt or solvate of any of both. Indeed, as will be depicted in the examples below, the ternary combinations including histidine provided good effects in terms of improving the rate of fatty acid beta-oxidation by the enzyme carnitine palmitoyltransferase I. These ternary compositions with at least histidine are, indeed cocktails of compounds that ultimately allow to increase the histidine plasma levels in the subject. Serine and cysteine are by several routes metabolized to obtain histidine, and carnosine is a source of histidine for being a dipeptide made up of the amino acids beta-alanine and histidine. NAC is a precursor of L-cysteine, it is converted into L-cysteine after ingestion.

In another particular embodiment of the pharmaceutical or dietary supplement composition according to the first aspect, the same comprises a therapeutically or nutraceutical effective amount of:
(a) Histidine, more in particular L-histidine, or a pharmaceutically or nutraceutical acceptable salt thereof or a solvate of any of both; cysteine, more in particular L-cysteine or a pharmaceutically or nutraceutical acceptable salt thereof or a solvate of any of both, and serine, more in particular L-serine, or a pharmaceutically or nutraceutical acceptable salt thereof or a solvate of any of both; or alternatively
(b) Histidine, more in particular L-histidine, or a pharmaceutically or nutraceutical acceptable salt thereof or a solvate of any of both; cysteine, more in particular L-cysteine, or a pharmaceutically or nutraceutical acceptable salt thereof or a solvate of any of both, and carnosine, more in particular L-carnosine, or a pharmaceutically or nutraceutical acceptable salt thereof or a solvate of any of both; or alternatively,
(c) cysteine, more in particular L-cysteine, or a pharmaceutically or nutraceutical acceptable salt thereof or a solvate of any of both, serine, more in particular L-serine, or a pharmaceutically or nutraceutical acceptable salt thereof or a solvate of any of both, and carnosine, more in particular L-carnosine, or a pharmaceutically or nutraceutical acceptable salt thereof or a solvate of any of both; or alternatively,
(d) histidine, more in particular L-histidine, or a pharmaceutically or nutraceutical acceptable salt thereof or a solvate of any of both, serine, more in particular L-serine, or a pharmaceutically or nutraceutical acceptable salt thereof or a solvate of any of both, and carnosine, more in particular L-carnosine, or a pharmaceutically or nutraceutical acceptable salt thereof or a solvate of any of both.

In another particular embodiment of the pharmaceutical or dietary supplement composition according to the first aspect, the composition comprises a therapeutically or nutraceutical effective amount of:
(a) cysteine, more in particular L-cysteine or a pharmaceutically or nutraceutical acceptable salt thereof or a solvate of any of both; histidine, more in particular L-histidine, or a pharmaceutically or nutraceutical acceptable salt thereof or a solvate of any of both; and serine, more in particular L-serine, or a pharmaceutically or nutraceutical acceptable salt thereof or a solvate of any of both; or alternatively
(b) cysteine, more in particular L-cysteine, or a pharmaceutically or nutraceutical acceptable salt thereof or a solvate of any of both; histidine, more in particular L-histidine, or a pharmaceutically or nutraceutical acceptable salt thereof or a solvate of any of both; and carnosine, more in particular L-carnosine, or a pharmaceutically or nutraceutical acceptable salt thereof or a solvate of any of both; or alternatively,
(c) cysteine, more in particular L-cysteine, or a pharmaceutically or nutraceutical acceptable salt thereof or a solvate of any of both, serine, more in particular L-serine, or a pharmaceutically or nutraceutical acceptable salt thereof or a solvate of any of both, and carnosine, more in particular L-carnosine, or a pharmaceutically or nutraceutical acceptable salt thereof or a solvate of any of both.

In another particular embodiment of the pharmaceutical or dietary supplement composition according to the first aspect, the composition comprises a therapeutically or nutraceutical effective amount of:
(a) NAC or a pharmaceutically or nutraceutical acceptable salt thereof or a solvate of any of both; histidine, more in particular L-histidine, or a pharmaceutically or nutraceutical acceptable salt thereof or a solvate of any of both; and serine, more in particular L-serine, or a pharmaceutically on nutraceutical acceptable salt thereof or a solvate of any of both; or alternatively
(b) NAC or a pharmaceutically or nutraceutical acceptable salt thereof or a solvate of any of both; histidine, more in particular L-histidine, or a pharmaceutically or nutraceutical acceptable salt thereof or a solvate of any of both; and carnosine, more in particular L-carnosine, or a pharmaceutically or nutraceutical acceptable salt thereof or a solvate of any of both; or alternatively,
(c) NAC or a pharmaceutically or nutraceutical acceptable salt thereof or a solvate of any of both, serine, more in particular L-serine, or a pharmaceutically or nutraceutical acceptable salt thereof or a solvate of any of both, and carnosine, more in particular L-carnosine, or a pharmaceutically or nutraceutical acceptable salt thereof or a solvate of any of both.

In a more particular embodiment of the first aspect of the invention, the composition comprises a therapeutically or nutraceutical effective amount of histidine or a pharmaceutically or nutraceutical acceptable salt thereof or a solvate of any of both; a therapeutically or nutraceutical effective amount of cysteine or a pharmaceutically or nutraceutical acceptable salt thereof or a solvate of any of both, a therapeutically or nutraceutical effective amount of serine or a pharmaceutically or nutraceutical acceptable salt thereof or a solvate of any of both, and a therapeutically or nutraceutical effective amount carnosine or a pharmaceutically or nutraceutical acceptable salt thereof or a solvate of any of both, together with one or more pharmaceutically or nutraceutical acceptable excipients or carriers.

More in particular, it comprises a therapeutically or nutraceutical effective amount of L-histidine or a pharmaceutically acceptable salt thereof or a solvate of any of both; a therapeutically or nutraceutical effective amount of L-cysteine or a pharmaceutically acceptable salt thereof or a solvate of any of both, a therapeutically or nutraceutical effective amount of L-serine or a pharmaceutically acceptable salt thereof or a solvate of any of both, and a therapeutically or nutraceutical effective amount of L-carnosine or a pharmaceutically acceptable salt thereof or a solvate of any of both, together with one or more pharmaceutically acceptable excipients or carriers.

In another more particular embodiment, the composition comprises a therapeutically or nutraceutical effective amount of L-histidine or a pharmaceutically acceptable salt thereof or a solvate of any of both; a therapeutically or nutraceutical effective amount of NAC or a pharmaceutically acceptable salt thereof or a solvate of any of both, a therapeutically or nutraceutical effective amount of L-serine or a pharmaceutically acceptable salt thereof or a solvate of any of both, and a therapeutically or nutraceutical effective amount of L-carnosine or a pharmaceutically acceptable salt thereof or a solvate of any of both, together with one or more pharmaceutically acceptable excipients or carriers.

In even a more particular embodiment, the composition comprises:
a therapeutically or nutraceutical effective amount of L-histidine monohydrochloride monohydrate;
a therapeutically or nutraceutical effective amount of L-cysteine hydrochloride;
a therapeutically or nutraceutical effective amount of L-serine; and
a therapeutically or nutraceutical effective amount of L-carnosine,
   together with one or more pharmaceutically or nutraceutical acceptable excipients or carriers.

In another even more particular embodiment, the composition comprises:
a therapeutically or nutraceutical effective amount of L-histidine monohydrochloride monohydrate;
a therapeutically or nutraceutical effective amount of NAC;
a therapeutically or nutraceutical effective amount of L-serine; and
a therapeutically or nutraceutical effective amount of L-carnosine,
   together with one or more pharmaceutically or nutraceutical acceptable excipients or carriers.

Particular therapeutic or nutraceutical amounts have been disclosed in previous paragraphs.

In a particular embodiment, the composition for human consumption comprises:
L-histidine monohydrochloride monohydrate in a therapeutically or nutraceutical effective amount giving a dose of 17 mg/Kg in human;
L-cysteine hydrochloride in a therapeutically or nutraceutical effective amount giving a dose of 39.7 mg/Kg in human;
L-serine in a therapeutically or nutraceutical effective amount giving a dose of 17 mg/Kg in human; and
L-carnosine in a therapeutically or nutraceutical effective amount giving a dose of 17 mg/Kg in human,
   together with one or more pharmaceutically acceptable excipients or carriers.

In another particular embodiment, the composition for human consumption comprises:
L-histidine monohydrochloride monohydrate in a therapeutically or nutraceutical effective amount giving a dose of 17 mg/Kg in human;
NAC in a therapeutically or nutraceutical effective amount giving a dose of 39.7 mg/Kg in human;
L-serine in a therapeutically or nutraceutical effective amount giving a dose of 17 mg/Kg in human; and
L-carnosine in a therapeutically or nutraceutical effective amount giving a dose of 17 mg/Kg in human,
   together with one or more pharmaceutically acceptable excipients or carriers.

In another particular embodiment, the composition for human consumption comprises:
L-histidine monohydrochloride monohydrate in a therapeutically or nutraceutical effective amount giving a dose of 7.5-20 mg/Kg in human;
L-cysteine hydrochloride in a therapeutically or nutraceutical effective amount giving a dose of 2-5 mg/Kg in human;
L-serine in a therapeutically or nutraceutical effective amount giving a dose of 1.3-3.3 mg/Kg in human; and
L-carnosine in a therapeutically or nutraceutical effective amount giving a dose of 4.5-10 mg/Kg in human,
   together with one or more pharmaceutically acceptable excipients or carriers.

In another particular embodiment, the composition for human consumption comprises:
L-histidine monohydrochloride monohydrate in a therapeutically or nutraceutical effective amount giving a dose of 7.5-20 mg/Kg in human;
NAC in a therapeutically or nutraceutical effective amount giving a dose of 2-5 mg/Kg in human;
L-serine in a therapeutically or nutraceutical effective amount giving a dose of 1.3-3.3 mg/Kg in human; and
L-carnosine in a therapeutically or nutraceutical effective amount giving a dose of 4.5-10 mg/Kg in human,
   together with one or more pharmaceutically acceptable excipients or carriers.

In another particular embodiment of the first aspect, the composition comprises a therapeutically or nutraceutical effective amount of a peptide, said peptide comprising a sequence of amino acids including at least three of histidine, cysteine, serine and carnosine, in any of their L- or D-configuration and combinations thereof (i.e., one compound in the D-isomer form and another in L-isomer form).

In another particular embodiment of the first aspect, the composition comprises a therapeutically or nutraceutical effective amount of a peptide, said peptide comprising a sequence of amino acids including cysteine and at least two of histidine, serine and carnosine in any of their L- or D-configuration and combinations thereof (i.e., one compound in the D-isomer form and another in L-isomer form). In another particular embodiment of the first aspect, the composition comprises a therapeutically or nutraceutical effective amount of a peptide, said peptide comprising a sequence of amino acids including cysteine, histidine, serine and carnosine in any of their L- or D-configuration and combinations thereof (i.e., one compound in the D-isomer form and another in L-isomer form).

In another particular embodiment of the first aspect, the composition comprises a therapeutically or nutraceutical effective amount of a peptide, said peptide comprising a sequence of amino acids including NAC and at least two of histidine, serine and carnosine, with the at least two of histidine, serine and/or carnosine in any of their L- or D-configuration and combinations thereof (i.e., one compound in the D-isomer form and another in L-isomer form). In another particular embodiment of the first aspect, the composition comprises a therapeutically or nutraceutical effective amount of a peptide, said peptide comprising a sequence of amino acids including NAC, histidine, serine and carnosine, with histidine, serine and carnosine in any of their L- or D-configuration and combinations thereof (i.e., one compound in the D-isomer form and another in L-isomer form).

These peptides with a sequence including at least three of the indicated compounds have, in another particular embodiment, a length of 3 to 50 amino acids, more in particular from 3 to 10 amino acids, and even more in particular a length selected from 3, 4, 5, 6, 7, 8, 9, and 10 amino acids.

In a more particular embodiment, the peptides are selected from tripeptides, tetrapeptides and pentapeptides.

In a preferred embodiment, the peptides in the composition are those comprising at least one histidine residue. In another preferred embodiment, the peptides in the composition are those comprising at least one cysteine or NAC residue. In another preferred embodiment, the peptides in the composition are those comprising at least one histidine residue and at least one cysteine residue, more preferably at least one histidine residue and at least one NAC residue.

In another particular embodiment, the tetrapeptides comprise at least three being selected from histidine, cysteine, serine and carnosine, being the additional amino acid any of the known by the skilled person in the art, including all the naturally occurring amino acids, in particular any selected from the 21 proteinogenic α-amino acids found in eukaryotes.

In another particular embodiment, the tetrapeptides comprise cysteine and at least two being selected from histidine, serine and carnosine, being the additional amino acid any of the known by the skilled person in the art, including all the naturally occurring amino acids, in particular any selected from the 21 proteinogenic α-amino acids found in eukaryotes. In another particular embodiment, the tetrapeptides comprise NAC and at least two being selected from histidine, serine and carnosine, being the additional amino acid any of the known by the skilled person in the art, including all the naturally occurring amino acids, in particular any selected from the 21 proteinogenic α-amino acids found in eukaryotes.

Amino acids in the sense of the present invention include: Alanine (A), Arginine (R), Asparagine (N), Aspartic acid (Aspartate, D), Cysteine (C), Glutamine (Q), Glutamic acid (E), Glycine (G), Histidine (H), Isoleucine (I), Leucine (L). Lysine (K), methionine (M), Phenylalanine (F), Proline (P), Pyrrolysine (O), Serine(S), Selenocysteine (U), Threonine (T), Tryptophan (W), Tyrosine (Y), and Valine (V). Modified amino acids include 2-Aminoadipic acid (Aad), 3-Aminoadicpic acid (bAad), beta-Alanine (bAla), 2-Aminobutyris acid (Abu), 6-Aminocaproic acid (Acp), 2-Aminoheptanoic acid (Ahe), 2-Aminoisobutyric acid (Aib), 2-Aminopimelic acid (Apm), 2,4-Diaminobutyric acid (Dbu), Desmosine, 2,2′-Diaminopimelic acid (Dpm), 2,3-Diaminoproprionic acid (Dpr), N-Ethylglycine (EtGly), N-Ethylasparagine (EtAsn), Hydroxylysine (Hyl), allo-Hydroxylysine (aHyl), 3-Hydroxiproline (3Hyp), 4-Hydroxyproline (4Hyp), Isodesmosine (Ide), allo-Isoleucine (alle), N-Methylisoleucine (Melle), 6-N-Methyllysine (MeLys), N-Methylvaline (Meval), Norvaline (Nva), Norleucine (Nle), and Ornithime (Orn). In several embodiments one amino acid may be replaced for another meanwhile at each selected position the hydrophobicity, aromaticity and basicity is maintained.

In another particular embodiment, the peptide comprises histidine, cysteine, serine and carnosine, and it is at least a pentapeptide, preferably a pentapeptide. In another particular embodiment, the peptide comprises histidine, NAC, serine and carnosine, and it is at least a pentapeptide, preferably a pentapeptide.

In all these possible peptides of the composition, the amino acid residues are in any order in the sequence read from N- to C-terminal end.

Once the composition is administered, the peptides comprised therein will be naturally hydrolysed by the common cell and body processes (i.e., hydrolases, proteases, etc.), and the single units will be delivered.

Adjustment of the dose of these peptides will vary, but in a particular embodiment it will be adjusted to give the above indicated doses for the single compounds as previously disclosed.

The preparation of all these peptides is according to the common practices known by the skilled person in the art.

All the pharmaceutical or dietary supplement compositions of the invention include the common excipients and/or carriers.

Examples of suitable pharmaceutically or nutraceutical acceptable excipients are solvents, dispersion media, diluents, or other liquid vehicles, dispersion or suspension aids, surface active agents, isotonic agents, thickening or emulsifying agents, preservatives, solid binders, lubricants and the like. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this invention.

The relative amounts of the active ingredient, the pharmaceutically or nutraceutical acceptable excipient, and/or any additional ingredients in a pharmaceutical or nutraceutical composition of the invention will vary, depending upon the identity, size, and/or condition of the subject treated and further depending upon the route by which the composition is to be administered.

Pharmaceutically or nutraceutical acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Excipients such as coloring agents, coating agents, sweetening, and flavoring agents can be present in the composition, according to the judgment of the formulator.

The pharmaceutical or nutraceutical compositions containing the combination of at least three of histidine, carnosine, serine and cysteine are for example, solid or liquid, and can be administered by any suitable route, for example, oral, parenteral, rectal, topical, intranasal, intraocular, intraperitoneal or sublingual route, for which they will include the pharmaceutically or nutraceutical acceptable excipients necessary for the formulation of the desired dosage form. More particularly, the pharmaceutical or dietary supplement composition of the first aspect of the invention is for example, solid or liquid, and can be administered by any suitable route, for example, oral, parenteral, rectal, topical, intranasal, intraocular, intraperitoneal or sublingual route, for which they will include the pharmaceutically or nutraceutical acceptable excipients necessary for the formulation of the desired dosage form.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, corn-starch, powdered sugar, and combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked polyvinylpyrrolidone) (crospovidone), sodium carboxymethyl starch (sodium starch glycolate), carboxymethyl cellulose, cross-linked sodium carboxymethyl cellulose (croscarmelose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and combinations thereof.

Exemplary binding agents include, but are not limited to, starch (e.g., corn-starch and starch paste); gelatin; sugars (e.g., sucrose, glucose, dextrose, dextrin, molasses, lactose, lactitol, mannitol); natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, polyvinylpyrrolidone), magnesium aluminium silicate (Veegum), and larch arabogalactan); alginates; polyethylene oxide; polyethylene glycol; inorganic calcium salts; silicic acid; polymethacrylates; waxes; water; alcohol; and combinations thereof.

Exemplary preservatives may include antioxidants, chelating agents, antimicrobial preservatives, antifungal preservatives, alcohol preservatives, acidic preservatives, and other preservatives. Exemplary antioxidants include, but are not limited to, alpha tocopherol, ascorbic acid, ascorbyl palmitate, ascorbyl stearate, ascorbyl oleate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and trisodium edetate.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and combinations thereof.

The preparation of all these pharmaceutical or nutraceutical compositions is according to the common practices known by the skilled person in the art.

As previously indicated, another aspect of the invention is a food product in solid or liquid form comprising the pharmaceutical or dietary supplement composition as defined in the first aspect. In a more particular embodiment, the food is a beverage. These food products or dietary supplements are, indeed, functional foods or supplements that provide a health benefit even in case no pathology is present. Thus, they are conceived as compositions that can prevent or reduce the risk of suffering from diseases, such as those related with fat deposition in tissues.

When a food product or a composition as disclosed in the first or second aspects and their embodiments was administered to mice with NAFLD, the animals were effectively treated reducing the fat accumulated in the liver. In the same way, the body weight due to fat mass was also reduced. Moreover, in an assay for the analysis of atherosclerosis risk, it was observed that the said risk was reduced when the animals received the composition of the invention. Furthermore, when the composition of the invention was administered to ovarectomized rat showing metabolic alterations associated to menopause, the values of the corresponding metabolic parameters got closer to those of control rats. Administration of the composition of the invention to NAFLD-induced mice decreased the expression of bacterial histidine utilization genes that were found to be increased in patients with NAFLD, and thus counteracts the microbiome dysregulation or dysbiosis. Thus, as a third aspect this pharmaceutical or dietary supplement composition, or the food product, is proposed for use in therapy.

In a particular embodiment of the third aspect of the invention, the composition of the first aspect or the food product of the second aspect is for use in the prevention and/or treatment of a disease selected from the group consisting of a fatty liver disease, obesity, overweight, atherosclerosis, dysbiosis and combinations thereof.

This can also be formulated as the use of the compositions or food product of the invention for the manufacture of a medicament for the prevention and/or treatment of a disorder or disease selected from the group consisting of a fatty liver disease, obesity, overweight, atherosclerosis, dysbiosis and combinations thereof. The present invention also relates to a method for the prevention and/or treatment of a disorder or disease selected from the group consisting of a fatty liver disease, obesity, overweight, atherosclerosis, dysbiosis and combinations thereof, comprising administering a therapeutically or nutraceutical effective amount of the composition or food product, together with pharmaceutically or nutraceutical acceptable excipients and/or carriers, in a subject in need thereof, including a human.

The term “obesity”, as used herein refers to an abnormal or excessive fat accumulation in a subject, in particular in a mammal, more in particular in a human, that presents a risk to health. The BMI associated to obesity in humans, particularly in adult humans, is of 30 or more. Obesity is frequently subdivided into 3 categories:
Class 1 obesrity: BMI of 30 to <35
Class 2 obesity: BMI of 35 to <40
Class 3 obesity: BMI of 40 or higher. Class 3 obesity is sometimes categorized as “severe” obesity.

BMI categories for children and teens are often based on sex- and age-specific BMI percentiles, whereas BMI categories for adults are based on BMI only. Children and teen obesity is subdivided in two categories:
Class 2 Obesity: BMI≥120% to <140% of the 95th percentile or BMI>35 to <40 kg/m2.
Class 3 Obesity: BMI>140% of the 95th percentile or BMI>40 kg/m2

The term “body Mass Index” or “BMI”, as used herein refers to a subject's weight in kilograms (or pounds) divided by the square of height in meters (or feet).

In a particular embodiment of the of the third aspect of the invention, the composition or food product is for use in the prevention and/or treatment of obesity in an adult human. In another particular embodiment of the of the third aspect of the invention, the composition or food product is for use in the prevention and/or treatment of obesity in a non-adult human. As well known by an expert in the field, a non-adult human is a human with less than 18 years, and an adult human is a human with 18 years or more.

In a particular embodiment of the of the third aspect of the invention, the composition or food product is for use in the prevention and/or treatment of class 1 obesity, class 2 obesity and/or class 3 obesity, particularly in an adult human. In a particular embodiment, it is for use in the prevention and/or treatment of class 1 obesity, particularly in an adult human. In another embodiment, it is for use in the prevention and/or treatment of class 2 obesity, particularly in an adult human. In another embodiment, it is for use in the prevention and/or treatment of class 3 obesity, particularly in an adult human. In another embodiment, it is for use in the prevention and/or treatment of class 1 or class 2 obesity, particularly in an adult human. In another embodiment, it is for use in the prevention and/or treatment of is class 2 or class 3 obesity, particularly in an adult human.

In a particular embodiment of the of the third aspect of the invention, the composition or food product is for use in the prevention and/or treatment of class 2 obesity and/or class 3 obesity, particularly in a non-adult human. In a particular embodiment, it is for use in the prevention and/or treatment of class 2 obesity, particularly in a non-adult human. In another embodiment, it is for use in the prevention and/or treatment of class 3 obesity, particularly in a non-adult human. In a particular embodiment of the of the third aspect of the invention, the composition or food product is for use in the prevention and/or treatment of class 2 obesity and class 3 obesity, particularly in a non-adult human. In a particular embodiment of the of the third aspect of the invention, the composition or food product is for use in the prevention and/or treatment of class 2 obesity or class 3 obesity, particularly in a non-adult human.

The term “overweight”, as used herein, refers to an abnormal or excessive fat accumulation in a subject, in particular a mammal, more in particular a human, that presents a risk to health, that is associated in humans to a BMI as defined above of between 25 and 30, in particular in adults, and 85^{th} percentile to less than the 95^{th} percentile for children and teens.

In a particular embodiment of the of the third aspect of the invention, the composition or food product is for use in the prevention and/or treatment of overweight, particularly in an adult human. In another particular embodiment of the of the third aspect of the invention, the composition or food product is for use in the prevention and/or treatment of overweight in a non-adult human.

The term “dysbiosis” refers to any condition in which the normal diversity or function of the body's microbiome is disrupted, particularly the microbiome of the gut.

In a more particular embodiment, the composition of the first aspect or the food product of the second aspect is for use in the prevention and/or treatment of a fatty liver disease selected from non-alcoholic fatty liver disease and alcoholic fatty liver disease.

In a more particular embodiment, it is for use in the prevention and/or treatment of a non-alcoholic fatty liver disease selected from non-alcoholic fatty liver (NAFL) with a simple steatosis and non-alcoholic steatohepatitis (NASH).

In another particular embodiment of the third aspect of the invention, the composition or food product is for use in the prevention and/or treatment of menopause-associated symptoms, in particular, the metabolic changes associated to menopause, in a female mammal, particularly in a woman.

This can also be formulated as the use of the compositions or food product of the invention for the manufacture of a medicament for the prevention and/or treatment of menopause-associated symptoms, in particular, the metabolic changes associated to menopause in a female mammal, particularly in a woman. The present invention also relates to a method for the prevention and/or treatment of menopause-associated symptoms, in particular, the metabolic changes associated to menopause, comprising administering a therapeutically or nutraceutical effective amount of the composition or food product, together with pharmaceutically or nutraceutical acceptable excipients and/or carriers, in a female mammal in need thereof, including a woman.

In another particular embodiment of the third aspect of the invention, the composition or food product is for use in the prevention and/or treatment of menopause-associated symptoms, in particular, the metabolic changes associated to menopause, and in the prevention and/or treatment of a disease selected from the group consisting of a fatty liver disease, obesity, overweight, atherosclerosis, dysbiosis, and combinations thereof, in a female mammal, particularly in a woman.

This can also be formulated as the use of the compositions or food product of the invention for the manufacture of a medicament for the prevention and/or treatment of menopause-associated symptoms, in particular, the metabolic changes associated to menopause, and for the prevention and/or treatment of a disease selected from the group consisting of a fatty liver disease, obesity, overweight, atherosclerosis, dysbiosis and combinations thereof, in a female mammal, particularly in a woman. The present invention also relates to a method for the prevention and/or treatment of menopause-associated symptoms, in particular, the metabolic changes associated to menopause, and for the prevention and/or treatment of a disease selected from the group consisting of a fatty liver disease, obesity, overweight, atherosclerosis, dysbiosis, and combinations thereof, comprising administering a therapeutically or nutraceutical effective amount of the composition or food product, together with pharmaceutically or nutraceutical acceptable excipients and/or carriers, in a female mammal in need thereof, including a woman.

The term “menopause-associated symptoms” as used herein, refers to metabolic, weight, cardiovascular, and musculoskeletal changes, genitourinary and skin atrophy; sexual dysfunction, and central-nervous-system-related disorders, that occur generally in a female mammal, particularly in a woman, as a consequence of menopause. The term “metabolic changes associated to menopause”, as used herein refers to changes in parameters associated to the metabolism that occur as a consequence of menopause in a female mammal, particularly in a woman. Non-limitative examples of said parameters include body weight, adipositiy, and insulin resistance. More particularly, said parameters include body weight, fat mass, lean mass, fat accumulation, adiposity index, insulin resistance.

In a particular embodiment, the metabolic changes associated to menopause are selected from the list consisting of increased body weight, increased adiposity, increased insulin resistance in a female mammal, particularly in a women, at menopause transition, menopause or postmenopause, as compared to before menopause, particularly before starting menopause transition.

In another particular embodiment, metabolic changes associated to menopause are selected from the list consisting of body weight gain, fat mass gain, lean mass loss, increased fat accumulation, increased adiposity index, increased insulin resistance, in a female mammal, particularly in a women, at menopause transition, menopause or postmenopause, as compared to before menopause, particularly before starting menopause transition.

In a particular embodiment, “before menopause” refers to 1 year, 2 years, 3 years, 4 years, preferably 1 year before starting menopause. In another particular embodiment, “before starting menopause transition” refers to 1 year, 2 years, 3 years, 4 years, preferably 1 year before starting menopause transition.

The term “menopause” as used herein, refers to the condition in a female mammal, preferably in a woman, characterized in that the ovaries are no longer ovulating or producing very few sex hormones. Clinically, natural menopause is defined as a 1-year period of amenorrhea following the final menstrual period (FMP) without any pathological or physiological cause.

The term “menopausal transition”, or ‘perimenopause’, is a defined period of time beginning with the onset of irregular menstrual cycles until the last menstrual period and is marked by fluctuations in reproductive hormones.

The term “postmenopause”, as used herein, refers to the time period after menopause has occurred, i.e. the time period starting at 12 months after the final menstrual period (FMP).

In another particular aspect, the invention relates to a non-therapeutic method for reducing body weight in a subject with overweight, that comprises administering the composition of the first aspect of the invention or the food product of the second aspect of the invention, to said subject, wherein the subject is in particular a mammal, even more in particular a human. In a particular embodiment, the doses of administration are as the doses disclosed in the first aspect of the invention for the composition of the invention. In another particular embodiment, the routes of administration are as those disclosed in the first aspect of the invention for the composition of the invention. In a particular embodiment, the human is an adult human. In another particular embodiment, the human is a non-adult human.

In a particular embodiment, reducing overweight in a subject with overweight involves reducing the BMI of the subject at least 5%, at least 10%, at least 15%, at least 20%, at least 25%, at least 30%, particularly at least 5%. In another particular embodiment, reducing overweight in a subject with overweight involves reducing the BMI of the subject at least 1, at least 2, at least 3, at least 4, at least 5, at least 6, at least 7 points, particularly at least 1 point.

As will be illustrated in the examples, a diet rich in fat and sugars when supplemented with the particular combination of four histidine, serine, cysteine, and carnosine, allowed the reduction of fat accumulated in the liver and in other body tissues.

Thus, also herewith disclosed for the first time is a combination comprising a therapeutically or nutraceutical effective amount of histidine or a pharmaceutically or nutraceutical acceptable salt thereof or a solvate of any of both; a therapeutically or nutraceutical effective amount of cysteine or a pharmaceutically or nutraceutical acceptable salt thereof or a solvate of any of both, a therapeutically or nutraceutical effective amount of serine or a pharmaceutically or nutraceutical acceptable salt thereof or a solvate of any of both, and a therapeutically or nutraceutical effective amount carnosine or a pharmaceutically or nutraceutical acceptable salt thereof or a solvate of any of both, together with one or more pharmaceutically or nutraceutical acceptable excipients or carriers. This combination was never disclosed and now it is proposed as dietary supplement for subjects with risk of suffering one or more of fatty liver disease, obesity and atherosclerosis. Any of the previous embodiments indicated for the first and the second aspects in relation with the amount (dose) of any of the compounds, as well as in relation with the isomers of the compounds do also applies to the indicated combination of the four ingredients.

Any of the previous embodiments indicated for the first and the second aspects in relation with the amount (dose) of any of the compounds, as well as in relation with the isomers of the compounds do also apply to the indicated combination of the four ingredients, wherein cysteine is substituted by NAC.

Throughout the description and claims the word “comprise” and variations of the word, are not intended to exclude other technical features, additives, components, or steps. Furthermore, the word “comprise” encompasses the case of “consisting of”. Additional objects, advantages and features of the invention will become apparent to those skilled in the art upon examination of the description or may be learned by practice of the invention. The following examples are provided by way of illustration, and they are not intended to be limiting of the present invention. Furthermore, the present invention covers all possible combinations of particular and preferred embodiments described herein.

### EXAMPLES

### 1.—Assay in Human Cohorts.

### 1.1.—Circulating Histidine Levels in Different Cohorts Depending on the Degree of Steatosis.

To obtain detailed information in hepatic steatosis for NAFLD patients, metabolic profiling of urine and plasma by 1H-NMR spectroscopy was performed. A metabolome-wide association study (MWAS) resulted in 124 metabolite signals in urine and 80 in plasma correlated with hepatic steatosis and associated clinical traits. Among some liver steatosis metabolites strongly correlated with NAFLD grades, a striking negative correlation with histidine in plasma and a significant decrease in urine were found. Plasma histidine was negatively correlated with liver steatosis for the 102 patients and 56 patients in the discovery studies, which was also confirmed in the two validation cohort studies with 313 and 283 patients. This 283 patients cohort study also showed a decrease of histidine levels in obese no steatosis patients compared to lean no steatosis patients. Circulatory histidine levels decrease when the degree of steatosis increase, confirming the negative correlation of histidine levels with liver steatosis.

### Example 1. Composition with Histidine, Serine, Cysteine and Carnosine as Supplement for Use in the Improvement of Hepatic Steatosis Clinical Symptoms

### Materials and Methods

### Animal Model and Diets

Forty-eight C57BL/6J male mice (Envigo, Sant Feliu de Codines, Barcelona, Spain), 6 weeks old at the beginning of the experiment, were used. Animals were housed in groups (4 mice per cage) under controlled conditions of temperature (22±2° C.) and humidity (55±10%), and on a 12-hour light/dark cycle with free access to food and water. Mice were left undisturbed to acclimate to the animal facility for one week. After the acclimatization period, animals were randomly divided into two experimental groups with different diets. Sixteen control mice were kept on a standard diet (D12328, Research Diets, New Brunswick, NJ) and 32 animals (NAFLD group) were fed with HFHFr diet (HFHC: D12331, Research Diets) supplemented with 23.1 g/L fructose and 18.9 g/L sucrose in the drinking water. Mice were kept on these diets for a period of 20 weeks in ad libitum conditions. These specific doses were determined based on previous studies and a calculation of dose translation from human to animal dosage. All experimental protocols were approved by the Animal Ethics Committee of the Technological Unit of Nutrition and Health of Eurecat (Reus, Spain) and the Generalitat de Catalunya approved all the procedures (10281). The experimental protocol followed the “Principles of Laboratory Care” guidelines and was carried out in accordance with the European Communities Council Directive (86/609/EEC).

For the last 4 weeks of the experiment (from the 16th to 20th week), NAFLD mice were randomly distributed into two groups: 16 mice were kept under the same fed conditions described before (HFHFr group), and 16 mice were exposed to histidine-related amino acids treatment (AA group). AA is a mix of the following compounds: 210 mg/kg of L-histidine monohydrochloride monohydrate (Merck, GmbH Germany), 490 mg/kg L-cysteine hydrochloride (Merck, GmbH Germany), 210 mg/kg L-serine (Merck, GmbH Germany) and 210 mg/kg L-carnosine 98% (Acros Organics, Geel, Belgium). Histidine, cysteine, serine and carnosine were diluted with drinking water with 23.1 g/L fructose and 18.9 g/L sucrose (vehicle). Fresh solutions were freshly prepared three times per week and prepared from stock powders and protected from light. Before being euthanized, 10 animals per group were randomly selected to perform an insulin challenge. They were intraperitoneally injected with 1 mU/g of insulin (n=5 per group) or saline (n=5 per group) and after 15 min, they were sacrificed.

Serum was obtained by centrifugation and stored at −80° C. for further analysis. Serum alanine aminotransferase (ALT) and aspartate aminotransferase (AST) were quantified by enzymatic colorimetric assays (QCA, Barcelona, Spain). Fasting insulinemia and glycemia were measured with the Mouse Insulin ELISA Kit (Mercodia, Uppsala, Sweden) and the Glucose Liquid Kit (QCA, Barcelona, Spain), respectively. Livers were rapidly collected, weighed and divided into two sections—the lobus hepatis sinister medialis was kept in formalin, and the remaining tissue was frozen in liquid nitrogen and stored at −80° C. until further analysis.

### Hepatic Fat Quantification

Hepatic lipids were extracted and quantified. Briefly, total lipids were extracted from 80-100 mg liver sections by adding 1 mL of hexane/isopropanol (3:2, v/v) and degassing with gas nitrogen. Then, they were left overnight under orbital agitation, at room temperature, protected from light. After extracting with 0.3 mL of Na₂SO₄ (0.47 M), the organic layer was separated and dried with gas nitrogen. Total lipids were quantified gravimetrically before emulsifying. Triglycerides and total cholesterol were measured using commercial enzymatic kits (QCA).

### Histological Evaluation

Liver portions fixed in buffered formalin (4% formaldehyde, 4 gr/L NaH2PO4, 6.5 gr/L Na₂HPO₄; pH 6.8) were cut at a thickness of 3.5 μm and stained with hematoxylin & eosin (H&E) and trichrome stain. Liver images (magnification 40×) were taken with a microscope (ECLIPSE Ti; Nikon, Tokyo, Japan) coupled to a digital sight camera (DS-Ri1, Nikon) and analyzed using ImageJ NDPI software (National Institutes of Health, Bethesda, MD, USA; https://imagej.nih.gov/ij, version 1.52a). To avoid any bias in the analysis, the study had a double-blind design, preventing the reviewers from knowing any data from the mice during the histopathological analysis. A General NAFLD Scoring System was established to diagnose mice with NAFLD/NASH. The key features of NAFLD and NASH were categorized as follows: steatosis was assessed by analyzing macrovesicular (0-3) and microvesicular steatosis (0-3) separately, followed by hepatocellular hypertrophy (0-3), which evaluates abnormal cellular enlargement, and finally giving a total score of 9 points of steatosis state. Inflammation was scored by counting cell aggregates (inflammatory foci). The score 0 to 3 depends on the grade of the feature. It is categorized as 0 (<5%), 1 (5-33%), 2 (34-66%) and 3 (>66%), and this scoring is used in each feature of steatosis and then added to the total steatosis score 23. Ballooning was not included in the scoring system, because only quantitative measures were considered for the rodent NAFLD score. It is important to highlight that hypertrophy is not a sign of cellular injury and slightly refers to an anomalous enlargement of the cells without recognizing the source of this enlargement.

### RNA Extraction and Quantitative Polymerase Chain Reaction

Homogenates from 8 livers per groups were used for total RNA extractions using TriPure reagent (Roche Diagnostic, Sant Cugat del Vallès, Barcelona, Spain) according to the manufacturer's instructions. RNA concentration and purity were determined using a nanophotometer (Implen GmbH, München, Germany). RNA was converted to cDNA using the High-Capacity RNA-to-cDNA Kit (Applied Biosystems, Wilmington, DE, USA). The cDNAs were diluted 1:10 before incubation with commercial LightCycler 480 Sybr green I master on a Lightcycler® 480 II (Roche Diagnostics GmbH, Manheim, Germany). Table 1 shows a list of used primers that were previously described in other studies and verified with Primer-Blast software (National Center for Biotechnology Information, Bethesda, MD, USA). As previously described, 36b4 was used as a housekeeping gene.

TABLE 1
Sequences of the used RT-PCR oligonucleotides.

| Primers | Forward | Reverse |
|---|---|---|
| Ppara | 5′-CCCTGTTTGTGGCTGCTATAATTT-3′ | 5′-GGGAAGAGGAAGGTGTCATCTG-3 |
| | (SEQ ID NO: 1) | (SEQ ID NO: 2)′ |
| Fasn | 5′-GCTGCGGAAACTTCAGGAAAT-3′ | 5′-AGAGACGTGTCACTCCTGGACTT-3′ |
| | (SEQ ID NO: 3) | (SEQ ID NO: 4) |
| Cd11c | 5′-ACGTCAGTACAAGGAGATGTTGGA-3′ | 5′-ATCCTATTGCAGAATGCTTCTTTACC-3′ |
| | (SEQ ID NO: 5) | (SEQ ID NO: 6) |
| Tnfa | 5′-AGGGTCTGGGCCATAGAACT-3′ | 5′-CCACCACGCTCTTCTGTCTAC-3′ |
| | (SEQ ID NO: 7) | (SEQ ID NO: 8) |
| II6 | 5′-AGTTGCCTTCTTGGGACTGA-3′ | 5′-TCCACGATTTCCCAGAGAAC-3′ |
| | (SEQ ID NO: 9) | (SEQ ID NO: 10) |
| II1a | 5′-CCAGAAGAAAATGAGGTCGG-3′ | 5′-AGCGCTCAAGGAGAAGACC-3′ |
| | (SEQ ID NO: 11) | (SEQ ID NO: 12) |
| F4/80 | 5′-CATAAGCTGGGCAAGTGGTA-3′ | 5′-GGATGTACAGATGGGGGATG-3′ |
| | (SEQ ID NO: 13) | (SEQ ID NO: 14) |
| Scd1 | 5′-AGATCTCCAGTTCTTACACGACCAC-3′ | 5′-GACGGATGTCTTCTTCCAGGTG-3′ |
| | (SEQ ID NO: 15) | (SEQ ID NO: 16) |
| Acc1 | 5′-GATGAACCATCTCCGTTGGC-3′ | 5′-CCCAATTATGAATCGGGAGTGC-3′ |
| | (SEQ ID NO: 17) | (SEQ ID NO: 18) |
| Cd36 | 5′-GAACCACTGCTTTCAAAAACTGG-3′ | 5′-TGCTGTTCTTTGCCACGTCA-3′ |
| | (SEQ ID NO: 19) | (SEQ ID NO: 20) |
| Fabp4 | 5′-TGAAAGAAGTGGGAGTGGGC-3′ | 5′-CGAATTCCACGCCCAGTTTG-3′ |
| | (SEQ ID NO: 21) | (SEQ ID NO: 22) |
| Cpt1a | 5′-CTCAGTGGGAGCGACTCTTCA-3′ | 5′-GGCCTCTGTGGTACACGACAA-3′ |
| | (SEQ ID NO: 23) | (SEQ ID NO: 24) |
| Cbs | 5′-GCAGCGCTGTGTGGTCATC-3′ | 5′-CATCCATTTGTCACTCAGGAACTT-3′ |
| | (SEQ ID NO: 25) | (SEQ ID NO: 26) |
| Cth | 5′-GGCTTCCTGCCTAGTTTCCA-3′ | 5′-AGTCAAAGGCATCCACCAAAG-3′ |
| | (SEQ ID NO: 27) | (SEQ ID NO: 28) |
| Acox | 5′- CTATGGGATCAGCCAGAAAG -3′ | 5′-ATGAGGTTGGCTTTCAGGAG-3′ |
| | (SEQ ID NO: 29) | (SEQ ID NO: 30) |
| Irf4 | 5′-CCCCAAAGCCCTCAGTCGTTGT-3′ | 5′-CAGCCACTCAGGCACCGCAC-3′ |
| | (SEQ ID NO: 31) | (SEQ ID NO: 32) |
| II-10 | 5′-AAGGCAGTGGAGCAGGTGAA-3′ | 5′-CCAGCAGACTCAATACACAC-3′ |
| | (SEQ ID NO: 33) | (SEQ ID NO: 34) |
| Atg5 | 5′-CGTGTATGAAAGAAGCTGATGC-3′ | 5′-ACGAAATCCATTTTCTTCTGGA-3′ |
| | (SEQ ID NO: 35) | (SEQ ID NO: 36) |
| Atg7 | 5′-ATGCCAGGACACCCTGTGAACTTC-3′ | 5′-ACATCATTGCAGAAGTAGCAGCCA-3′ |
| | (SEQ ID NO: 37) | (SEQ ID NO: 38) |
| Mtus1 | 5′-ACAGAAGATGGATCGCGTTATG-3′ | 5′-CCCCGTGACTCACTGAAGG-3′ |
| | (SEQ ID NO: 39) | (SEQ ID NO: 40) |
| LepR | 5′-TCCAGGAGAGATGCTCACACTTT-3′ | 5′-TGCGTGGAACAGGTTTGAAA-3′ |
| | (SEQ ID NO: 41) | (SEQ ID NO: 42) |
| Sec24b | 5′-GACCCGAGAAGGCGCTTT-3′ | 5′-TTTGCCAACCCAAATGTAGAAA-3′ |
| | (SEQ ID NO: 43) | (SEQ ID NO: 44) |
| Cebpb | 5′-ATCGACTTCAGCCCGTACCT-3′ | 5′-CCGTAGTCGTCGGAGAAGAG-3′ |
| | (SEQ ID NO: 45) | (SEQ ID NO: 46) |
| 36bB4 | 5′-AGTCCCTGCCCTTTGTACACA-3′ | 5′-CGATCCGAGGGCCTCACTA-3′ |
| | (SEQ ID NO: 47) | (SEQ ID NO: 48) |

### Protein Extraction and Western Blot Analysis

Approximately 20 mg of liver was homogenized with Tyssuelyser LT (Qiagen, Hilden, Germany) for 50 seconds in 300 UL lysis buffer (8 mmol/L NaH2PO4, 42 mmol/L Na2HPO4, 1% SDS, 0.1 mol/L NaCl, 0.1% NP40, 1 mmol/L NaF, 10 mmol/L sodium orthovanadate, 2 mmol/L PMSF, and 1% protease inhibitor cocktail 1 (Millipore Sigma, Darmstadt, Germany)). The protein extracts were quantified by the standardized BCA method (Bio-Rad Protein Assay; BioRad, Hercules, CA, USA). Protein extracts (20-25 μg) were electrophoretically separated on 10% SDS-PAGE and electroblotted to nitrocellulose membranes (Li-cor biosciences, NE, USA). Efficient protein transfer was monitored by the Ponceau-S stain. Next, membranes were blocked (5% BSA) at room temperature and probed with specific primary antibodies (diluted 1:1000) overnight at 4° C. in 1% BSA: total Akt (4685) (CST, Danvers, MA, USA), phospho-Akt (Ser473) (4060) (CST) and β-Actin (Santa Cruz Biotechnology, Inc; Dallas, TX, USA). Thereafter, infrared fluorescent secondary antibodies anti-rabbit 680, anti-rabbit 800 and anti-mouse 680 (LI-COR Biosciences, Lincoln, NE, USA; 926-32211, 926-68071 and 926-68070, respectively) were used for detection and quantified using ImageJ.

### Statistical Analysis

Statistical analyses were performed using GraphPad Prism 9 software (Graph-Pad Soft-ware, La Jolla, CA, USA). Data are presented as mean±SEM. Data distribution was analyzed by the Shapiro-Wilk normality test. Differences between the two groups were determined using an unpaired t-test (two-tailed, 95% confidence interval). One-way analysis of variance (ANOVA) was conducted to examine differences between three groups. A p-value below 0.05 was considered statistically significant. Statistical analyses were performed using GraphPad Prism 9 software (Graph-Pad Software, La Jolla, CA, USA). Data are presented as mean±SEM. Data distribution was analyzed by the Shapiro-Wilk normality test. Differences between the two groups were determined using an unpaired t-test (two-tailed, 95% confidence interval). One-way analysis of variance (ANOVA) was conducted to examine differences between three groups. A p-value below 0.05 was considered statistically significant.

### Results

### Specific Amino Acids Combination Reduced Liver Injury and Liver Features of NAFLD

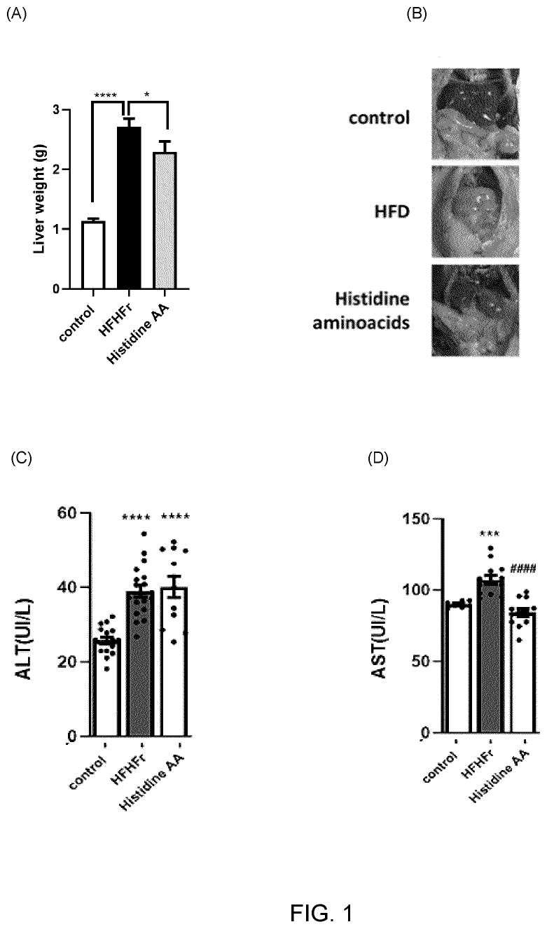

After 4-weeks of specific amino acids treatment (AA) composed of histidine, serine, cysteine and carnosine, animals were sacrificed, and livers weight were analyzed. AA supplemented group showed a tendency to decrease liver weight compared to HFHFr animals, which significantly increased liver weight compared to the control group (FIG. 1 (A)). These differences found between AA and HFHFr groups in liver weight were not correlated with differences in solid or liquid intake (data not shown). Moreover, a macroscopic observation was performed showing whitish and fatty liver in HFHFr animals compared to the control group, whereas AA treatment partially recovered a healthy liver appearance in comparison with the HFHFr group (FIG. 1 (B)). The AA group presented a significantly decreased level of AST compared to HFHFr animals. However, ALT did not show any significant change in the AA group concerning the HFHFr group counterparts, which showed a significant increase compared to the control group in both transaminases (FIGS. 1 (C) and (D)).

### AA Supplementation Decreased Hepatic Lipid Content and Liver Steatosis

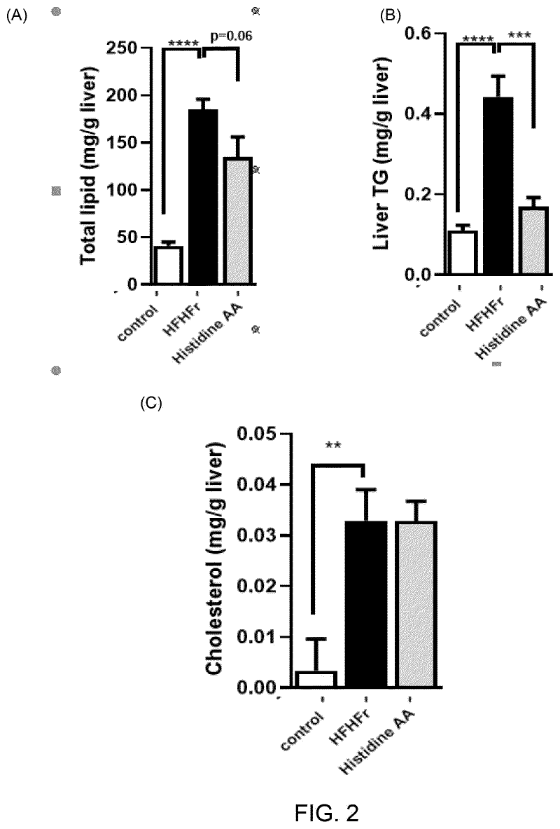

After the assessment of liver status, this study analyzed the hepatic liver content and the characteristic features of hepatic steatosis. Significant increases in the hepatic levels of total lipid content, total cholesterol and triglycerides were observed in the HFHFr group compared to the control group. Interestingly, AA supplemented animals showed a significant decrease in hepatic triglycerides levels compared to the HFHFr group (FIG. 2 (B)), but this effect was not reflected in total cholesterol level in the liver (FIG. 2 (C). However, total hepatic lipid levels showed a slight decrease in AA group in comparison with the HFHFr group (FIG. 2 (A)).

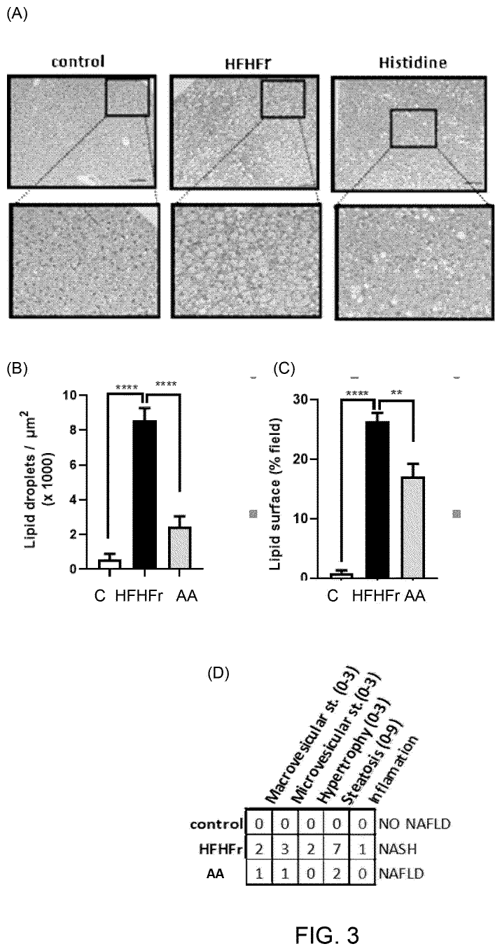

Consistent with liver state assessment and biochemical analysis, liver histology analysis showed that HFHFr group developed pronounced liver steatosis by the presence of macro- and microvesicular steatosis with increased hypertrophy, which trigger nuclear displacement. Significantly, treatment with AA supplementation ameliorated liver steatosis, indeed microvesicular steatosis was especially reduced, the occupied surface by macrovesicular steatosis was also reduced and hypertrophy was diminished lowering nucleus displacement (FIG. 3 (A)). Consequently, a significant reduction of lipid droplet amount and lipid surface was observed (FIGS. 3 (B) and (C)). Diagnosis of mice with NAFLD/NASH was performed through the establishment of a General NAFLD Scoring System, which corroborated the significant amelioration of different parameters analyzed in animals treated with AA supplementation in comparison with HFHFr group (FIG. 3 (D)).

In order to evaluate whether this combination of specific amino acids modulated the levels of proinflammatory markers, the expression levels of some representative inflammatory genes were analyzed. Related to prior results of this study, a marker of macrophage infiltration, F4/80, was significantly downregulated in AA supplemented animals compared to HFHFr group, which showed significant upregulated levels of this gene compared to control group. Similar results were observed in TNFα mRNA levels, which were meaningfully increased in HFHFr animals compared to control group, even though TNFα expression was significantly downregulated in AA supplemented group compared to their counterparts (data not shown).

### Beneficial Effects of AA Supplementation Did Not Involve Lipogenesis, or Lipid Transport Pathways

To discern which metabolic pathways could be involved in NAFLD improvement after AA supplementation, analysis of gene expression related to lipid metabolism were carried out, such as hepatic lipogenesis, lipid transport. The HFHFr group showed a significant increase in lipid transport-related genes Cd36 and Fabp4. In AA group is not seen any effect in these two genes related to lipid transport, maintaining high levels of expression of these genes compared to control group. Significant changes were observed in mRNA level of the gene involved in de novo hepatic lipogenesis, Scd1 when were compared to control and HFHFr groups. Others de novo hepatic lipogenesis genes Acc1 and Fasn did not show significant differences between control and HFHFr groups. After AA treatment no differences were found in novo hepatic lipogenic genes levels compared to the HFHFr group, except for Acc1 where the AA group showed a significant increase with respect to HFHFr group.

### AA Supplementation Reduced Hepatic Inflammation Associated to NAFLD

To evaluate whether supplementation with AA modulated the levels of proinflammatory markers, the expression levels of some representative inflammatory genes were assessed. As expected, F4/80 mRNA levels, a marker of macrophage infiltration, were significantly upregulated in the HFHFr group compared to control mice. In contrast, AA treatment significantly corrected this upregulation of F4/80 expression. Similar significant results were observed in the expression of Tnfα which the increased levels in HFHFr group was corrected in AA group, and an interesting statistical tendency was found in II1a gene expression with a significant increase in the HFHFr group compared to control group, however AA fails to lover significantly the levels of II1a. No considerable results were shown in both 116 and IL 10 levels. However, the expression of Cd11c showed a significant increase in HFHFr and AA group compared to the control group, although no differences were found between HFHFR and AA mice.

### AA Supplementation Increased Antioxidant Defense and Antitumoral Activity in NAFLD

An analysis of the genes related to autophagy, immune response, antioxidative defense, antitumoral activity, and vesicle traffic was carried out. Focus on autophagy, no differences were found in both genes between control, HFHFr and AA mice. However, when talking about genes related to antioxidant activity, such as CBS or CTH, significant results were found. Between control and HFHFr no difference was found, but in AA mice the expression of both genes was increased significantly compared to the HFHFr group. In the case of antitumoral activity-related genes, Mtus1 showed a significant increase in AA mice compared to the HFHFr group. Regarding the genes related to immune response, no differences were found in IRF4 expression. However, in Cebpb expression a significant increase was observed in the AA group compared to HFHFr group. To end, the gene related to vesicle traffic, Sec24b, showed a significant increase in AA mice compared to HFHFr mice.

### AA Supplementation Reduced Hepatic Insulin Resistance Associated to NAFLD

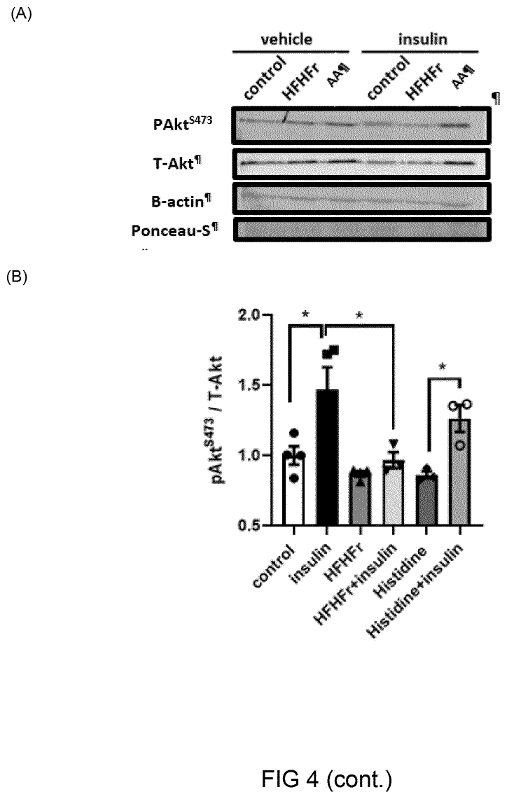

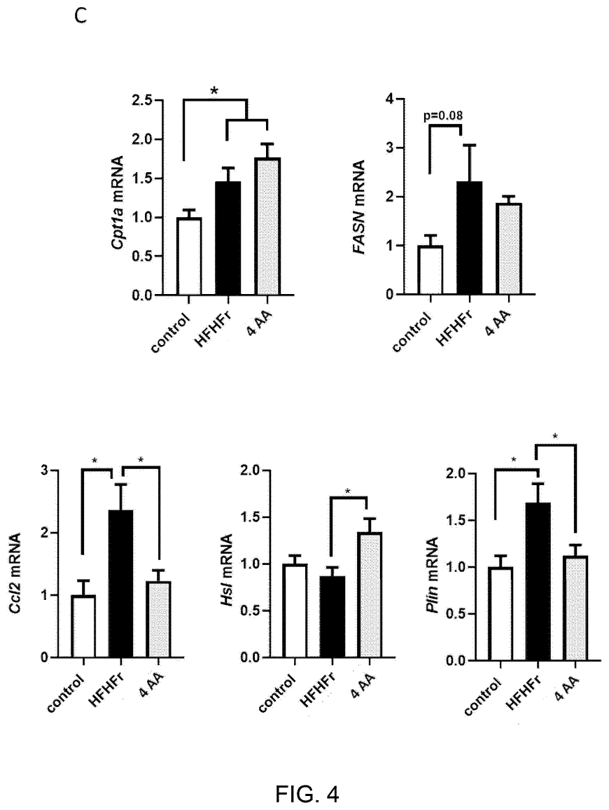

Considering that impaired hepatic insulin signaling plays an important role in NAFLD development, in order to evaluate insulin resistance, animals were challenged with an intraperitoneal insulin bolus determining Akt activation (phosphorylation on Serine 473) or saline. As expected, the control group showed a significant Akt phosphorylation (pAktS473) in insulin-challenged mice compared to vehicle-injected mice. This fact was not observed in the HFHFr group, indicating hepatic insulin resistance in these animals. In contrast, the AA-supplemented group showed a significant improvement in insulin signaling, increasing the phosphorylation of Akt significantly in insulin-challenged mice compared to vehicle-injected AA mice. Data are depicted in FIG. 4, where in (A) a representative Western blot analysis with Akt activation (pAktS473), total Akt protein levels (T-Akt), housekeeping β-actin levels and protein loading with Ponceau-S membrane staining. In (B) the densitometry analysis of phosphorylated and total Akt ratio is shown.

In addition, analysis of hepatic expression of key genes after the quaternary treatment of fatty acid beta-oxidation (carnitine palmitoyltransferase, Cpt1a), inflammation (chemokine (C-C motif) ligand 2, CCL2), lipolysis (hormone-sensitive, HSL; and lipin, PLIN) and lipogenesis (fatty acid synthase, FASN), showed a tendency to increase of Cpt1a, an important reduction of hepatic inflammation (Ccl2) and an increase of the lipolysis (higher expression of the lipase Hsl and lower levels of the surrounding lipid droplet protein Plin) (FIG. 4 (C)). In contrast, no changes were observed at the lipogenesis (Fasn). Data are expressed in FIG. 4 as mean±SEM; n=10-12 per condition. *p<0.05.

### AA Supplementation Increases Levels of Amino Acids in the Liver in NAFLD

In order to analyze the effect of the treatment based on amino acids on the liver, an assessment by LC/MS was performed in serum with the aim of quantifying the amino acids participants in the treatment. In the case of histidine and serine separately no differences were found between groups. When the levels of amino acids were put together a tendency to increase the concentration of these amino acids was observed but it was not significant. It could happen due to the tissue bioaccumulation of the amino acids. Therefore, an analysis of these amino acids was performed in liver homogenates of the different groups through LC/MS to prove this hypothesis. The results obtained showed a significant decrease in histidine and serine levels in the HFHFr group compared to the control group. Moreover, in the AA group, a strong tendency to increase histidine and serine levels was observed compared to the HFHFr group. Alanine, which is an amino acid that is part of Carnosine and can be produced through Carnosine synthetase, showed a significant decrease in the HFHFr group compared to the control group. Furthermore, a significant increase in AA mice compared to HFHFr mice. Glycine is an amino acid that is related to serine and GSH metabolism and an increase in its levels may be due to serine administration. A decreased glycine levels were found in the HFHFr group compared to the control group. Moreover, glycine levels were recovered in AA mice regarding HFHF mice glycine levels.

### Example 2. The Supplement of AA Reduces the Risk of Atherosclerosis

Atherosclerosis is a complex disorder that displays many of the characteristics of a chronic inflammatory process. Atherosclerotic cardiovascular disease remains the leading cause of morbidity and mortality worldwide and obesity increases the risk of atherosclerosis and death. Elucidating the mechanisms behind differences between obese individuals with and without atherosclerosis could reveal therapeutic targets for treating the harmful cardiovascular consequences of obesity as an alternative or adjunct to weight-loss programs, which are known to have limited long-term success. One of the most compelling clinical challenges of our time is the increasing prevalence of obesity and its detrimental effects on the cardiovascular system. Obesity influences inflammation and the pathophysiological processes involved in atherosclerotic disease development. Obesity and overweight are accompanied by unfavourable blood lipid profile patterns. Dyslipidemia is a major risk factor for coronary artery disease. Among obese patients, the estimated prevalence of hypertriglyceridemia is twice as high as in non-obese individuals. In addition, the atherogenic combination of hypertriglyceridemia with high LDL and low HDL is more prevalent in obese and overweight patients.

### Serum and Blood Analysis

Serum fasting glucose, total cholesterol, triglycerides (QCA, Barcelona, Spain) were analyzed by enzymatic colorimetric assays. Serum fasting insulinemia was analyzed using a mice insulin ELISA kit (Mercodia, Uppsala, Sweden) and HDL and LDL levels in serum were analyzed using EnzyChrom™ AF HDL and LDL/VLDL Assay Kit (BioAssay System, Hayward, CA, USA).

Sixteen control mice were kept on a standard diet (D12328, Research Diets, New Brunswick, NJ, USA) and 32 animals (atherogenic group) were fed with HFHFr diet (HFHC: D12331, Research Diets) supplemented with 23.1 g/L fructose and 18.9 g/L sucrose in the drinking water. Mice were kept on these diets for 20 weeks in ad libitum conditions. For the last 4 weeks of the experiment (from the 16th to 20th week), atherogenic mice were randomly distributed into two groups: 16 mice were kept under the same fed conditions described before and the other 16 were treated with the combination of histidine amino acids as explained above in Example 1.

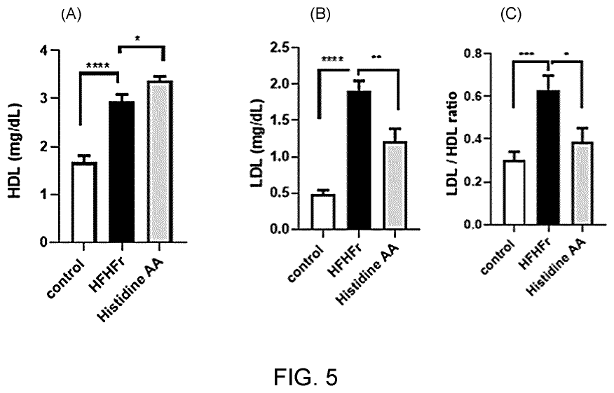

In an assay with n=48 mice it was also determined which were the circulating levels of high density lipoproteins (HDL), low-density lipoproteins (LDL) and the ratio LDL/HDL. Data are depicted in FIG. 5, where it is seen that the group receiving the supplement (Histidine AA) presented higher levels of HDL (FIG. 5 (A) in relation to the mice receiving HFHFr diet, with a meaningful decreased LDL in serum in relation of the mice fed with the HFHFr diet (FIG. 5 (B)), being the ratio LDL/HDL within a mean value as the control (FIG. 5 (C)). The data confirm that the supplement allows a reduction in the known as “bad cholesterol” (LDL) in levels an increase of in the known as “good cholesterol” (HDL) diminishing the risk of atherosclerosis.

### Example 3. The Supplement of AAs Reduces Visceral Fat and Obesity Parameters

Adipose tissue is singularly heterogenic. Subcutaneous adipose tissue (scWAT) constitutes the great majority and visceral adipose (vWAT) tissue a lesser part of the total body adipose stores, but an excess of vWAT is nevertheless a more severe risk than an excess of scWAT, which has been reported to exert a protective influence. In this regard, obesity is defined as an excessive accumulation or abnormal distribution of body fat, which can also be complicated by associated diseases, such as type 2 diabetes mellitus, hepatic steatosis, cardiovascular diseases, stroke, dyslipidemia, hypertension and certain types of cancer among others. Even though obesity is commonly caused by an excessive energy consumption in relation to energy expenditure, its aetiology is complex and includes genetic, physiologic, environmental, social, psychological, and economic factors, which interact to promote the development of obesity. This excessive energy availability promotes the expansion of adipose tissue depots, which is mediated by the growth of adipocyte size (hypertrophy) an increased in the number of adipocytes (hyperplasia).

In an assay with a same mice number as in Example 1, it was also determined the body weight, body weight gain among other parameters to elucidate the capacity of the supplemented (AA) to reduce obesity risk and the visceral fat.

### Materials and Methods

### Histological Analysis of EWAT and BAT

EWAT and BAT portions fixed in buffered formalin (4% formaldehyde, 4 gr/L NaH2PO4, 6.5 gr/L Na2HPO4; pH 6.8) were cut at a thickness of 5 μm and stained with hematoxylin & eosin (H&E). BAT IWAT and EWAT images (magnification 4×) were taken with a microscope (ECLIPSE Ti; Nikon, Tokyo, Japan) coupled to a digital sight camera (DS-Ri1, Nikon) and analyzed using ImageJ NDPI software (National Institutes of Health, Bethesda, MD, USA; https://imagej.nih.gov/ij, version 1.52a). To avoid any bias in the analysis, the study had a double-blind design, preventing the reviewers from knowing any data from the mice during the histopathological analysis. Area quantification of adipocytes was analyzed by the Adiposoft plugin to assess EWAT state between groups. Lipid droplets quantification in BAT was analyzed by the Droplet Finder plugin.

### RNA Extraction and Quantitative Polymerase Chain Reaction

According to the manufacturer's instructions, homogenates from EWATs, IWATs and BATs were used for total RNA extractions using the TriPure reagent (Roche Diagnostic, Sant Cugat del Vallès, Barcelona, Spain). RNA concentration and purity were determined using a nanophotometer (Implen GmbH, München, Germany). RNA was converted to cDNA using the High-Capacity RNA-to-cDNA Kit (Applied Biosystems, Wilmington, DE, USA). The cDNAs were diluted 1:10 before incubation with commercial LightCycler 480 Sybr green I master on a Lightcycler® 480 II (Roche Diagnostics GmbH, Manheim, Germany). Table 2 shows a list of used primers that were previously described in other studies and verified with Primer-Blast software (National Center for Biotechnology Information, Bethesda, MD, USA). 36b4 was used as a housekeeping gene.

### BAT Temperature Measurements

The temperature surrounding BAT was visualized using a high-resolution infrared camera (FLIR Systems) and analyzed with a specific software package (FLIR-Tools-Software, FLIR; Kent, UK), as previously described 79. For each image, the area surrounding BAT was delimited and the average temperature of the skin area was calculated as the average of 3 pictures/animal.

TABLE 2
Sequences of the oligonucleotides
used in the RT-PCR.

| Primers | Forward | Reverse |
|---|---|---|
| AdipoQ | AAGGGAGAGAAAGGAGATGC | TACACATAAGCGGCTTCTCC |
| | (SEQ ID NO: 49) | (SEQ ID NO: 50) |
| Ccl2 | ATTGGGATCATCTTGCTGGT | CCTGCTGTTCACAGTTGCC |
| | (SEQ ID NO: 51) | (SEQ ID NO: 52) |
| Cpt1a | CTCAGTGGGAGCGACTCTTC | GGCCTCTGTGGTACACGACAA |
| | A (SEQ ID NO: 23) | (SEQ ID NO: 24) |
| Cpt1b | CGAGGATTCTCTGGAACTGC | GGCCTCTGTGGTACACGACAA |
| | (SEQ ID NO: 53) | (SEQ ID NO: 54) |
| Dio2 | AGAGTGGAGGCGCATGCT | GGCATCTAGGAGGAAGCTGTT |
| | (SEQ ID NO: 55) | C (SEQ ID NO: 56) |
| Dio3 | CTACGTCATCCAGAGTGGCA | CTGTTCATCATAGCGCTCCA |
| | (SEQ ID NO: 57) | (SEQ ID NO: 58) |
| Fasn | GCTGCGGAAACTTCAGGAAA | AGAGACGTGTCACTCCTGGAC |
| | T (SEQ ID NO: 3) | TT (SEQ ID NO: 4) |
| Fgf21 | CCTCTAGGTTTCTTTGCCAA | AAGCTGCAGGCCTCAGGAT |
| | CAG (SEQ ID NO: 59) | (SEQ ID NO: 60) |
| Glut4 | AAAAGTGCCTGAAACCAGAG | TCACCTCCTGCTCTAAAAGG |
| | (SEQ ID NO: 61) | (SEQ ID NO: 62) |
| Hsl | TCCTGGAACTAAGTGGACGC | CAGACACACTCCTGCGCATAG |
| | AAG (SEQ ID NO: 63) | AC (SEQ ID NO: 64) |
| Lep | CTCCATCTGCTGGCCTTCTC | CATCCAGGCTCTCTGGCTTCT |
| | (SEQ ID NO: 65) | (SEQ ID NO: 66) |
| LepR | TCCAGGAGAGATGCTCACAC | TGCGTGGAACAGGTTTGAAA |
| | TTT (SEQ ID NO: 41) | (SEQ ID NO: 42) |
| Pgc1a | AGCCGTGACCACTGACAACG | GCTGCATGGTTCTGAGTGCTA |
| | AG (SEQ ID NO: 67) | AG (SEQ ID NO: 68) |
| Ppary | CACAATGCCATCAGGTTTGG | GCTGGTCGATATCACTGGAGA |
| | (SEQ ID NO: 69) | TC (SEQ ID NO: 70) |
| Prdm16 | CAGCACGGTGAAGCCATTC | GCGTGCATCCGCTTGTG |
| | (SEQ ID NO: 71) | (SEQ ID NO: 72) |
| Tfam | CACCCAGATGCAAAACTTTC | CTGCTCTTTATACTTGCTCAC |
| | AG (SEQ ID NO: 73) | AG (SEQ ID NO: 74) |
| Tmem26 | ACCCTGTCATCCCACAGAG | TGTTTGGTGGAGTCCTAAGGT |
| | (SEQ ID NO: 75) | C (SEQ ID NO: 76) |
| Ucp1 | ACTGCCACACCTCCAGTCAT | CTTTGCCTCACTCAGGATTGG |
| | T (SEQ ID NO: 77) | (SEQ ID NO: 78) |
| 36b4 | AGTCCCTGCCCTTTGTACAC | CGATCCGAGGGCCTCACTA |
| | A (SEQ ID NO: 47) | (SEQ ID NO: 48) |

### Results

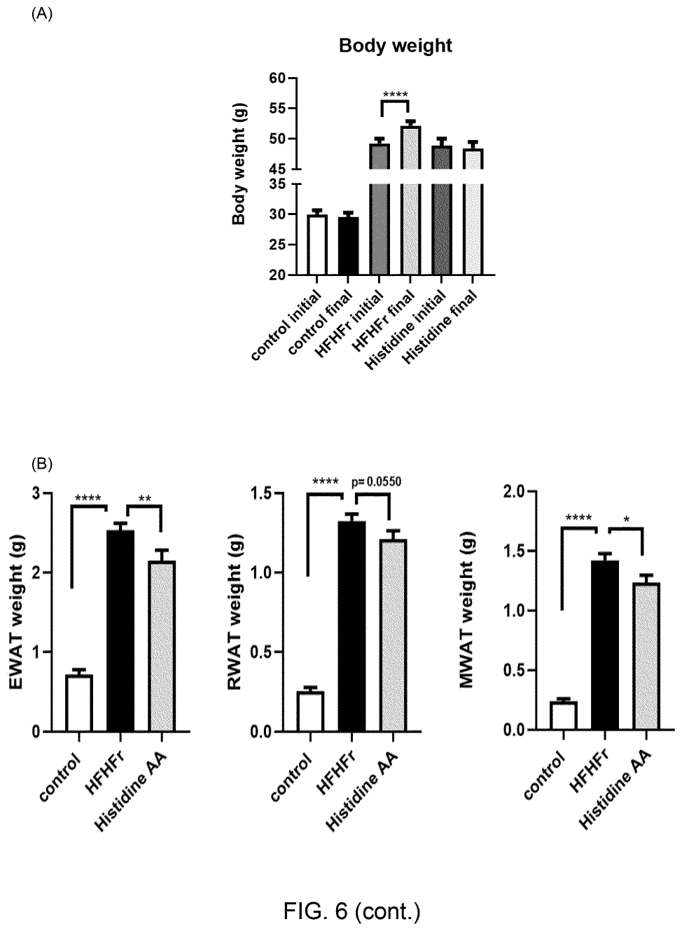

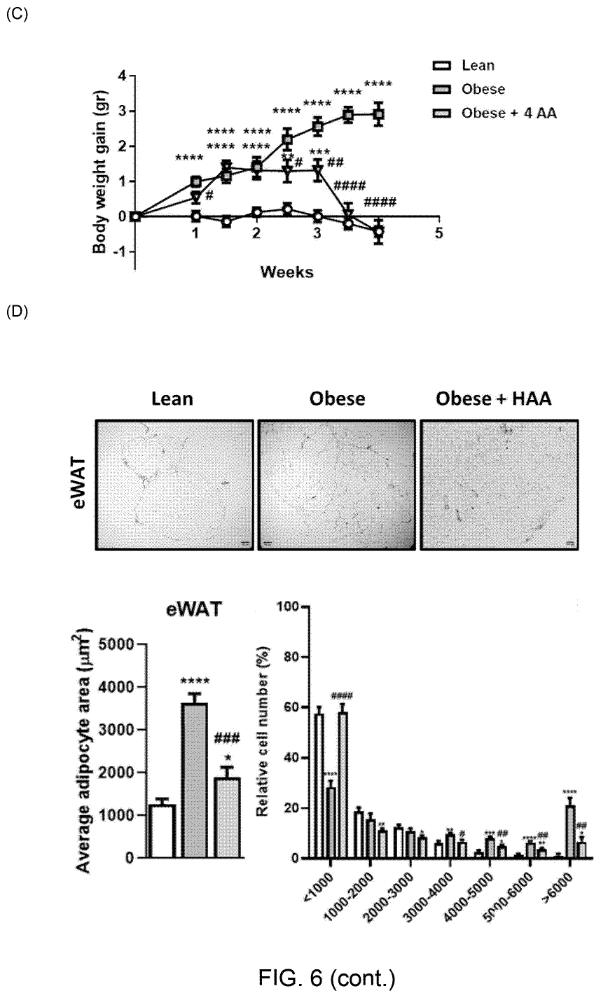

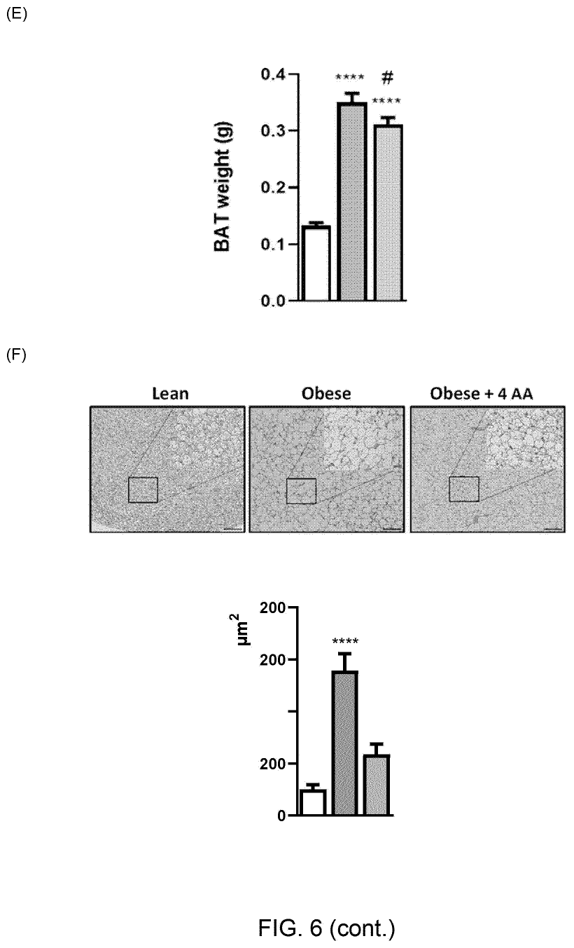

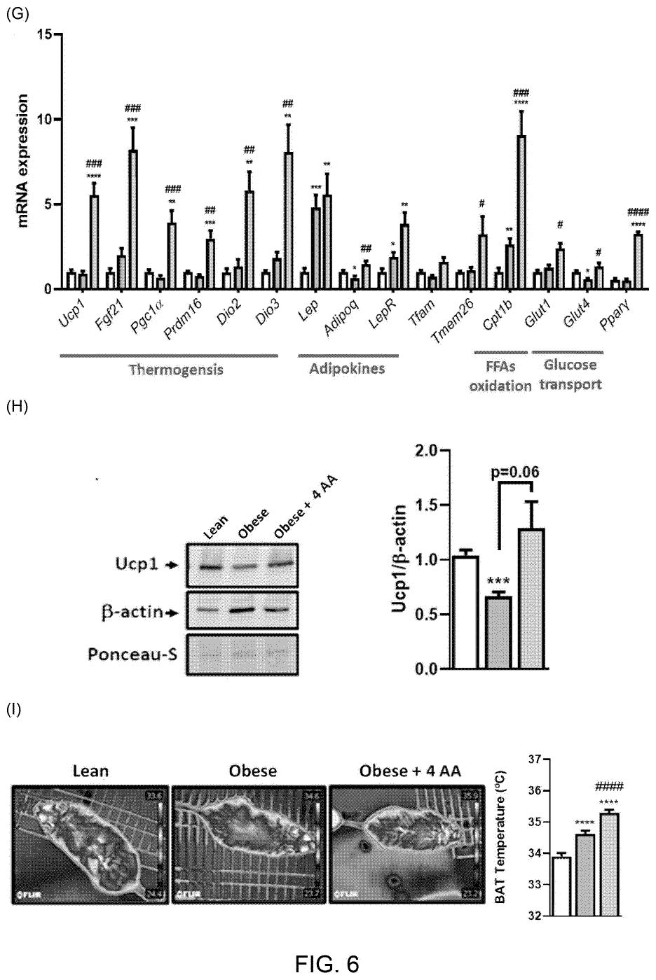

As can be seen in the data depicted in FIG. 6, the body weight of the mice fed with the HFHFr diet supplemented with the quaternary combination of the invention (histidine, cysteine, serine and carnosine) did not alter their initial and final weight after 4 weeks of treatment, contrary to the mice only fed with HFHFr diet, in which the final body weight was significatively higher than the initial one (FIG. 6 (A)). In addition, obese animals treated with the quaternary combination of the invention stopped the body weight gain at the second week of the treatment (FIG. 6 (C)). Moreover, when the epididymal white adipose tissue (EWAT), retroperitoneal WAT (RWAT), and the mean weight adipose tissue was determined, those mice fed with the HFHFr diet supplemented with the quaternary combination had a reduction in the amount of fat in relation to the mice with no supplementation (FIG. 6 (B)). Reduction in the hypertrophic visceral adipose tissues by quaternary combination treatment, was corroborated at microscopic level by a reduction in the adipocyte size, with an increased number of smaller adipocytes and lower number of larger adipocytes (FIG. 6 (D). Besides, the quaternary combination treatment also modulates the brown adipose tissue (BAT) reducing the hypertrophy in this adipose depot, together with a reduction in the brown adipocytes size (FIG. 6 (E)). These effects in the BAT were accompanied by an increase in the mRNA expression ok key thermogenic genes (Ucp1, Fgf21, Pgc1a, Prdm16, Dio2, and Dio3), an increase of the insulin-sensitizer adipokine (Adipoq) as well other genes related with the fatty acid oxidation (Cpt1b) and glucose uptake (Glut1 and Glut4) (FIG. 6 (G). Increased mRNA levels of Ucp1 after treatment with the quaternary combination were corroborated at protein level, indicating a higher capacity to burn fatty acids (FIG. 6 (H). Indeed, this capacity was evaluated by thermography observing a higher temperature in the BAT area of the animals treated with the quaternary combination (FIG. 6 (I).

### Example 4. In Vitro Assay

An in vitro assay was also carried out using the hepatocyte cell line (HepG2) to test the effect of different combinations of the supplemented amino acids in hepatic expression of key genes of fatty acid beta-oxidation (carnitine palmitoyltransferase, Cpt1a), inflammation (chemokine (C-C motif) ligand 2, CCL2), lipolysis (hormone-sensitive, HSL; and lipin, LPIN) and lipogenesis (fatty acid synthase, FASN). Gene expression was determined by qPCR as previously indicated (see materials and methods in example 1). Table 3 shows a list of the primers used. The Cpt1a enzyme is located in the outer membrane, and it is the key enzyme in the carnitine-dependent transport across the mitochondrial inner membrane. Its deficiency results in a decreased rate of fatty acid beta-oxidation.

TABLE 3
Sequences of the oligonucleotides
used in the RT-PCR.

| Primers | Forward | Reverse |
|---|---|---|
| CPT1a | GACGGCTATGGTGTGTCGTA | TCCAGCCCAGCACATGAAC |
| | (SEQ ID NO: 79) | (SEQ ID NO: 80) |
| CCL2 | AGCAGCAAGTGTCCCAAAGA | GGTGGTCCATGGAATCCTGA |
| | (SEQ ID NO: 81) | (SEQ ID NO: 82) |
| HSL | CAAGTGTGTCAGCGCCTATG | CAAGTGTGTCAGCGCCTATG |
| | CT (SEQ ID NO: 83) | CT (SEQ ID NO: 84) |
| PLIN | TGCCAGAAACAGCATCAG | CAAATTCCGCAGTGTCTCT |
| | (SEQ ID NO: 85) | (SEQ ID NO: 86) |
| FASN | TGCGTGGCCTTTGAAATGTG | CTCCATGTCCGTGAACTGCT |
| | (SEQ ID NO: 87) | (SEQ ID NO: 88) |
| RPLP0 | ATGGCAGCATCTACAACCCT | AGGACTCGTTTGTACCCGTT |
| | (SEQ ID NO: 89) | (SEQ ID NO: 90) |

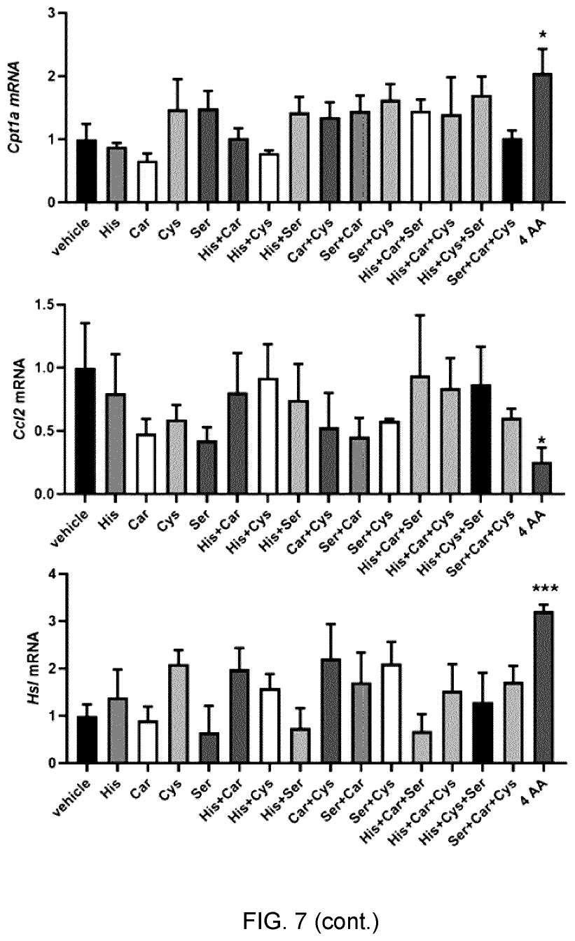

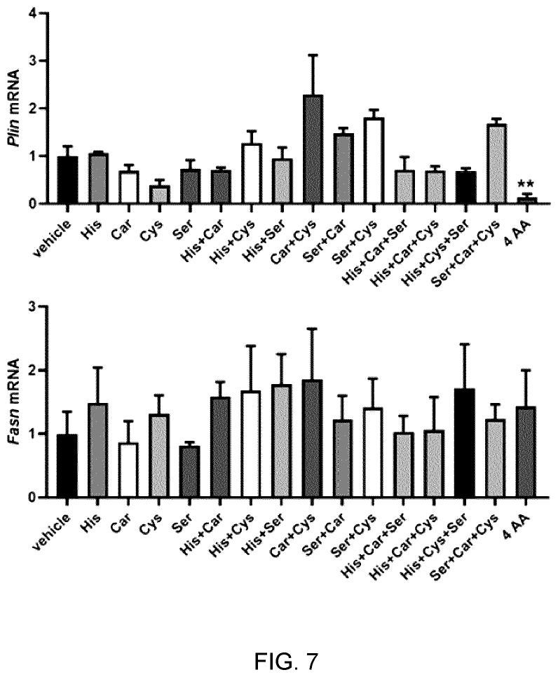

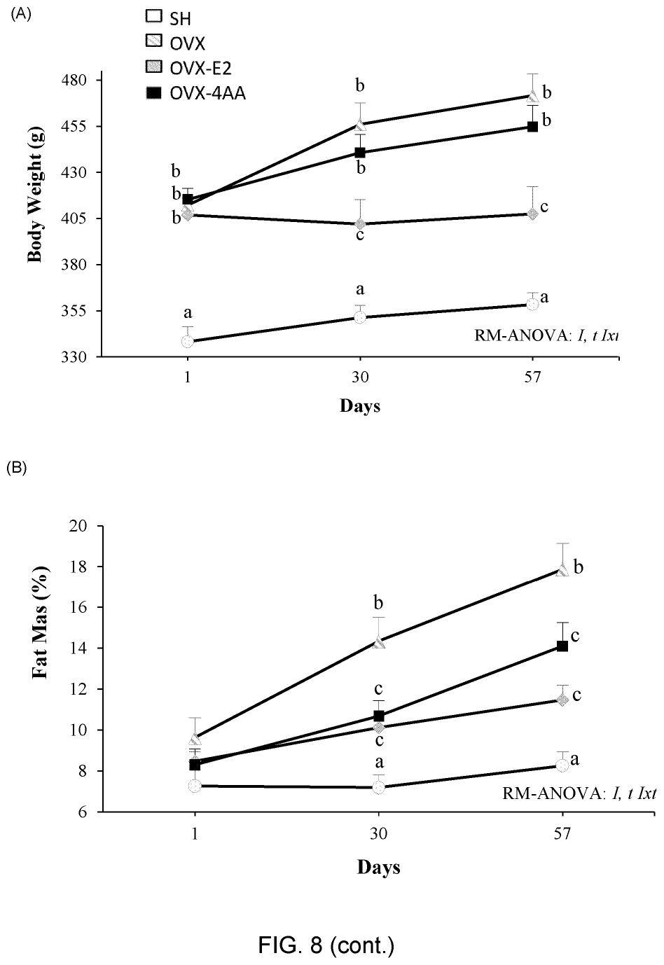

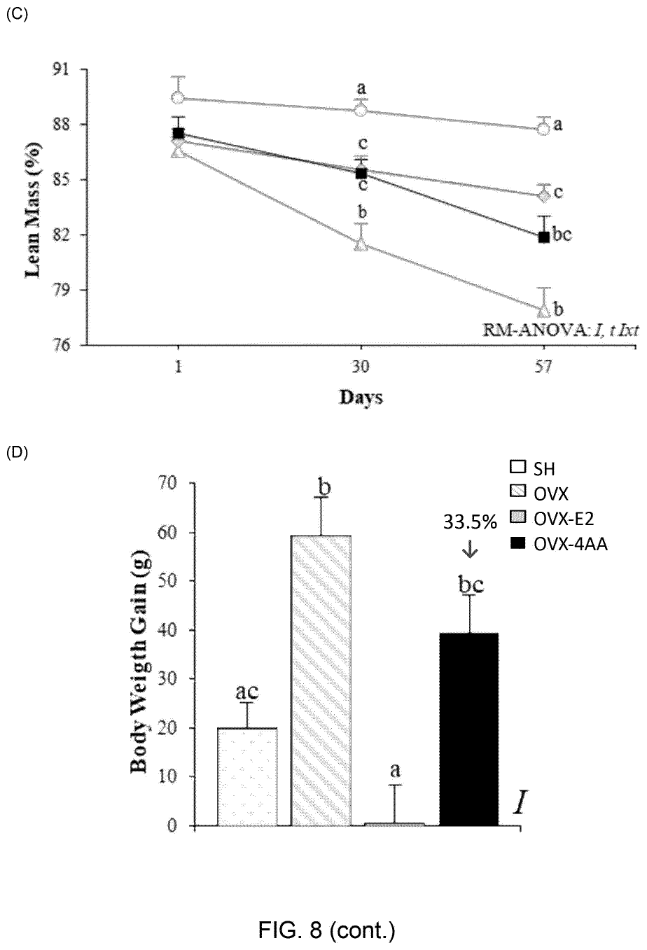

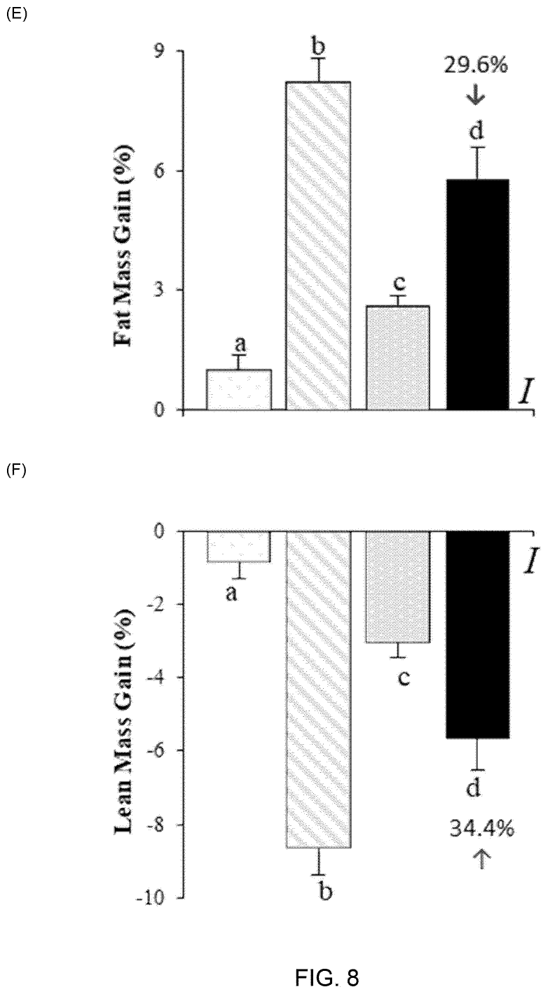

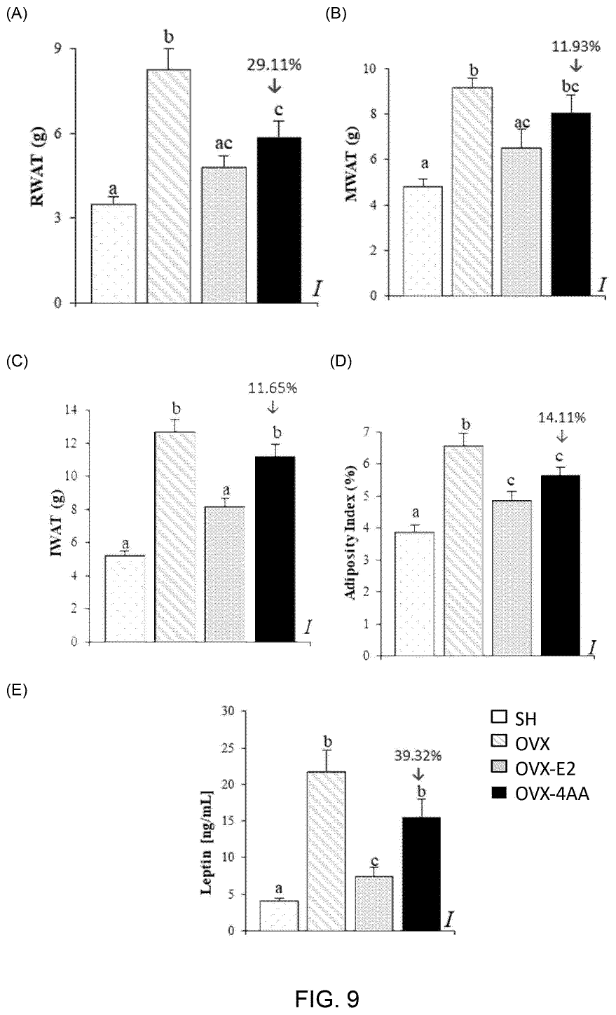

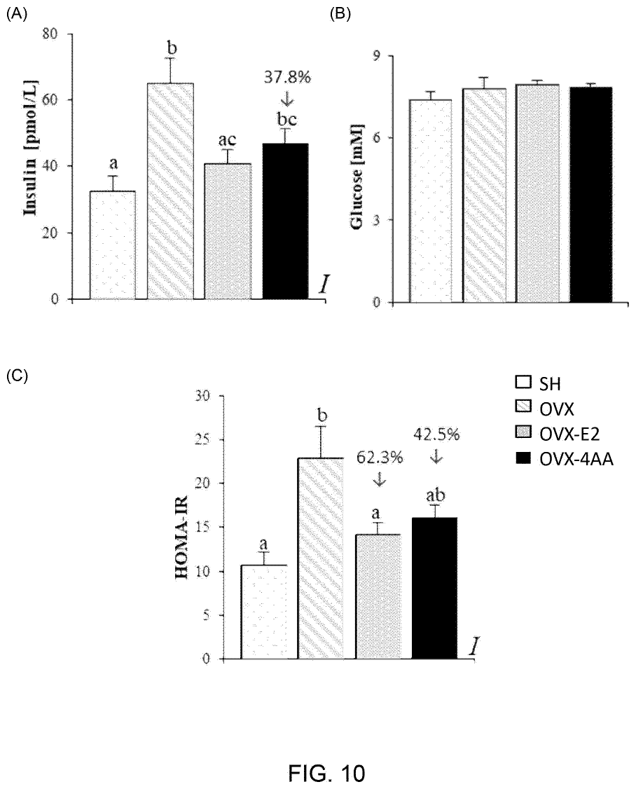

Data are depicted in FIG. 7, wherein the level of expression (mRNA) is indicated for any of the supplemented media. The tested supplements were histidine (His), carnosine (Car), cysteine (Cys), serine (Ser), histidine+carnosine (His+Car), histidine+serine (His+Ser), histidine+cysteine (His+Cys), carnosine+cysteine (Car+Cys), serine+carnosine (Ser+Car), serine+cysteine (Ser+Cys), histidine+carnosine+serine (His+Car+Ser), histidine+cysteine+serine (His+Cys+Ser), histidine+carnosine+cysteine (His+Car+Cys), serine+carnosine+cysteine (Ser+Car+Cys), and the quaternary histidine+cysteine+serine+carnosine (4 AA) . . . . The tested concentrations were as follows: Histidine 10 mM, carnosine 5 mM, cysteine 5 mM and serine 5 mM. These concentrations were conserved with the different combinations. Data are expressed in FIG. 7 as mean±SEM; n=3-4 per condition.

As depicted in FIG. 7, the best performance was with the supplement with 4AA. Ternary combinations were also effective (all having histidine in the combination) in relation to the control group, showing an increase of the effect in relation to the expression of the cited enzyme caused by the single amino acid. Of note is also that the ternary combination including histidine, serine and cysteine shows a pronounced increase. Taken altogether, these data allow to affirm that a combination of at least three of histidine, cysteine, serine, carnosine, in particular the ternary and quaternary combinations including histidine, surprisingly increased the level of the analyzed enzyme, as proof of their positive role in those diseases or disorders involving fat accumulation. Noteworthy is also that the quaternary combination performed synergistically better, even in the presence of carnosine, which compound demonstrated a trend to reduce the effect when added with some of the other amino acids.

### Example 5. The Supplement of AAs Ameliorates Metabolic Alterations Associated to Menopause

### Materials and Methods

Forty 24-week-old rats were ovarioectomized following the procedure described in Koebele, S. V. and Bimonte-Nelson, H. A, 2016. Ten additional rats of the same age were sham-operated. Between two and three weeks after the surgery, rats were randomly assigned to four experimental groups for 8 weeks:
SHAM, simulated operation
OVX: OVX rats that received daily a placebo
OVX-E2: OVX rats that received biweekly an estrogen treatment (17β-estradiol) by subcutaneous injections. OVX-E2 rats received biweekly subcutaneous injections of 25 μg/kg of body weight of 17β-oestradiol (Sigma-Aldrich, St. Louis, MO) via a carrier solution of corn oil.
OVX+4AA: OVX rats that were supplemented daily with the 4AA at doses 105 mg/kg/bw for histidine, carnosine, serine and 245 mg/kg/bw for cysteine.

Body composition analyses: Lean and fat mass measurements (in grams) were performed without anaesthesia at the beginning and at the end of the study using an EchoMRI-700™ device (Echo Medical Systems, L.L.C., Houston, USA). The measurements were performed in triplicate under ad libitum conditions and at 8.00 am and the results were expressed as a percentage of body weight.

Adiposity index: The adiposity index was calculated as the sum of the weights of the IWAT, MWAT and RWAT depot weights (in grams) and was expressed as a percentage of body weight.

Serum leptin levels: Serum leptin levels were determined with a rat ELISA kit (EZRL-83K/Millipore, Barcelona, Spain).

Glucose and insulin levels: Glucose and insulin levels were analyzed using and a glucose enzymatic colorimetric kit (998282/QCA, Barcelona, Spain) and a rat insulin ELISA kit (10-1250-01/Mercodia, Upssala, Sweden).

HOMA-IR analyses: The homeostasis model assessment-estimated insulin resistance (HOMA-IR) was calculated using the following the formula: (Glucose_Insulin)/22.5 as described previously (Matthews et al., 1985).

### Results

Previous research carried out with ovariectomized (OVX) rats showed that these animals display increased body weight, adiposity and insulin resistance impairment, compared to sham rats (control).

The results disclosed herein show that treatment with the quaternary histidine+cysteine+serine+carnosine (4 AA) combination is effective in ameliorating metabolic alterations associated to menopause.

### Example 6 . . . . Associations of Plasma Histidine and Steatosis Degree with the Gut Microbiota and Hut Genes in Humans

### Materials and Methods

### Metagenomic Analysis

For metagenomics analyses, those microbial families, genera, or species having less than 10 counts in 10% of the simples were excluded. Differential abundance analyses for taxa associated with the circulating histidine levels, hepatic steatosis or HAA treatment were performed using the analysis of compositions of microbiomes with bias correction (ANCOM-BC) methodology. It considers the bias due to differential sampling fractions across samples by adding a sample-specific offset to a linear regression model, that is estimated from the observed data. The linear regression model in log scale is analogous to log-ratio transformation to consider the compositional nature of metagenomics datasets, while the offset term serves as the bias correction. All models in humans for age, sex, BMI, and country were adjusted. P-values were adjusted for multiple comparisons using a Sequential Goodness of Fit as implemented in the “SGOF” R package. Unlike FDR methods, which decrease their statistical power as the number ohutG, hutH, hutI, and hutU, respectively.

### Extraction of Fecal Genomic DNA and Whole-Genome Shotgun Sequencing (Metagenomics)

Faecal shotgun sequencing data was generated for n=73 patients from the discovery cohort. Briefly, total DNA was extracted from frozen human stools using the QIAamp DNA Stool Mini kit (Qiagen), with slight modifications by adding a bead-beating step. Quantification of DNA was performed with a Qubit 3.0 fluorometer (Thermo Fisher Scientific, USA), and 3 ng of extracted DNA for each sample were prepared using the Bioscientific PCR free library kit according to the manufacturer's instructions and sequenced on a Hiseq 2500 (Illumina) with 2×150 pair-end chemistry. The obtained input fastq files were decompressed, filtered and 3′ ends-trimmed by quality, using prinseq-lite-0.20.4 program and overlapping pairs were joined using FLASH-1.2.11. Fastq files were then converted into fast files, and human host reads were removed by mapping the reads against the reference human genome (GRCh38.p11, December 2013) using Bowtie 2 with end-to-end and very sensitive options. Next, functional analyses were carried out by assembling the non-host reads into contigs by MEGAHIT v1.1.2 and mapping those reads against the contigs with Bowtie 2. Reads that did not assemble were appended to the contigs. Next, Prodigal v2.6.342 was used for predicting codifying regions. Functional annotation was carried out with HMMER against the Kyoto Encyclopedia of Genes and Genomes (KEGG) database, version 2016 to obtain the functional subcategory, route and annotation of the genes. The filtering of the best annotations and the assignment of the orf annotation to every read were carried out using R 3.1.0 which was also used to count the aligned reads and to add the category and its coverage, and finally to build abundance matrices. Taxonomic annotation was implemented with Kaiju v1.6.2 on the human-free reads using a greedy mode. Addition of lineage information, counting of taxa and generation of an abundance matrix for all samples were performed using the package R.

Bacterial Genomic DNA Isolation, 16S rRNA Sequencing and Analysis (Metagenomics)

Bacterial genomic DNA was isolated from mice fecal samples using the MBP DNA Soil extraction kit. Genomic DNA was normalized to 5 ng/μL with EB (10 mM Tris-HCl) and libraries were performed. Briefly, following a first PCR and clean-up using KAPA Pure Beads (Roche Catalogue No. 07983298001) a second PCR master mix was made up using P7 and P5 of Nextera XT Index Kit v2 index primers (Illumina Catalogue No. FC-131-2001 to 2004). Following the PCR reaction, the libraries were quantified using the Quant-iT dsDNA Assay Kit, high sensitivity kit (Catalogue No. 10164582) and run on a FLUOstar Optima plate reader. Libraries were pooled and run on a High Sensitivity D1000 ScreenTape (Agilent Catalogue No. 5067-5579) using the Agilent Tapestation 4200 to calculate the final library pool molarity. The pool was run on an Illumina MiSeq instrument using MiSeq® Reagent Kit v3 (600 cycle) (Illumina Catalogue FC-102-3003) following the Illumina recommended denaturation and loading recommendations which included a 20% PhiX spike in (Phix Control v3 Illumina Catalogue FC-110-3001). The raw data was analysed locally on the MiSeq using MiSeq reporter. For the 16S sequence analysis, The LotuS 1.36 used pipeline in short amplicon mode with default quality filtering. Raw 16S rRNA gene reads were quality filtered to ensure a minimum length of 170 bp, not more than eight homonucleotides, no ambiguous bases, average quality>=27 and an accumulated error below 0.5 and a dereplication filter set to “6:1,3:2”. Cleaned reads were clustered into amplicon sequencing variants (ASVs) using DADA2, chimeric ASVs were removed using the DADA2 de novo chimera check. Remainder phiX reads were filtered by mapping ASVs against the phiX reference genome. ASV taxonomy was assigned using the LotuS2 LCA algorithms against Silva 138.1 reference database. On average 5500+3108 reads could be assigned to each sample that were of cyanobacterial origin. Further data analysis was conducted with R statistical language Version 3.00 (The R Foundation, https://www.r-project.org/), employing the rtk software or all data normalizations. The 16S raw data can be found at https://1drv.ms/u/s!ApVezdktX3allZVJXYWok9s2ghgfvg?e=VDyr2c.

### Primer Design for HutH and HutG Microbiota Functions

Several sequences from prokaryotes species were uploaded from databases to ARDEP program in order to design degenerated primers that could amplify in conserved regions for HutH and HutG genes and cover all the species used for the primer design. Based on the k-mer algorithm, primer length is set as k, and all sequences are divided into k-mers. The analysis platform allows for sequence length statistics, length filtering, and redundancy removal to be performed on the sequence database using tools located in the “Sequence Processing” section. After performing sequence database quality control, ARDEP for primer design could be run in the “Primer Processing” section, and a list of different possibilities of primer sets was obtained. The “Covered Taxonomy Calculator” option was used to count the sequence number of taxonomy and functional group covered by primer sets. The basic primer properties were considered (Tm, GC %, primer length) choosing the best option.

### Results

Microbiota-host interactions potentially contribute to the development of metabolic diseases, and molecular networks linking the gut microbiome to hepatic steatosis have recently been unravelled.

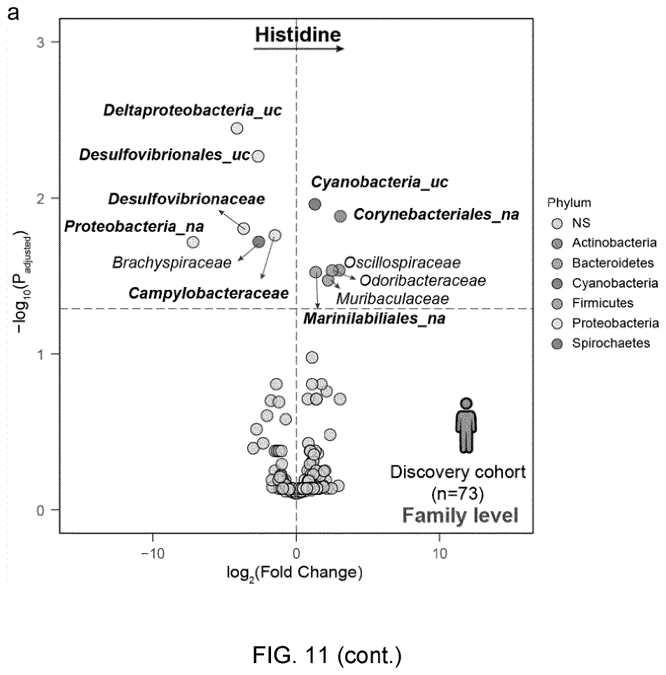

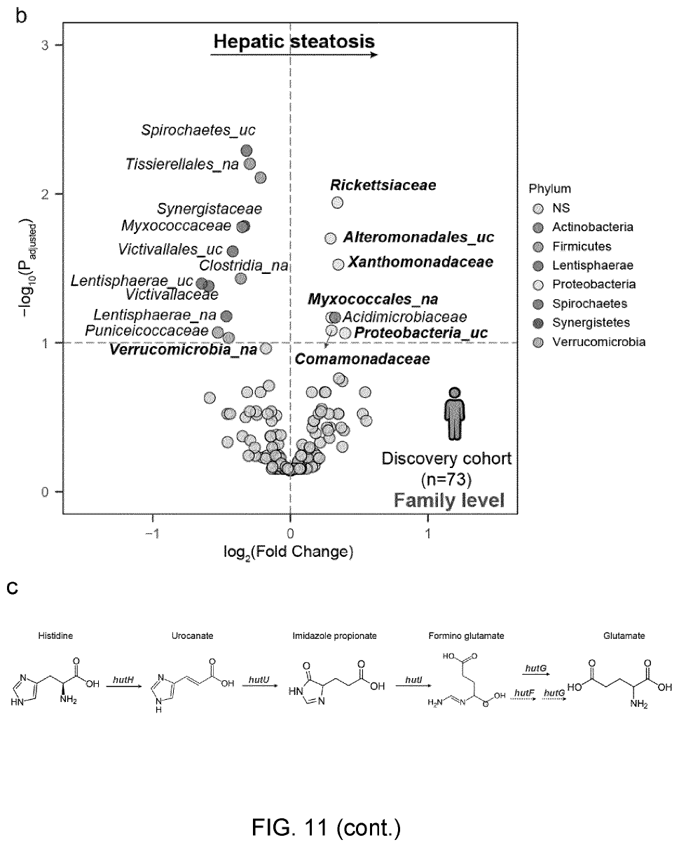

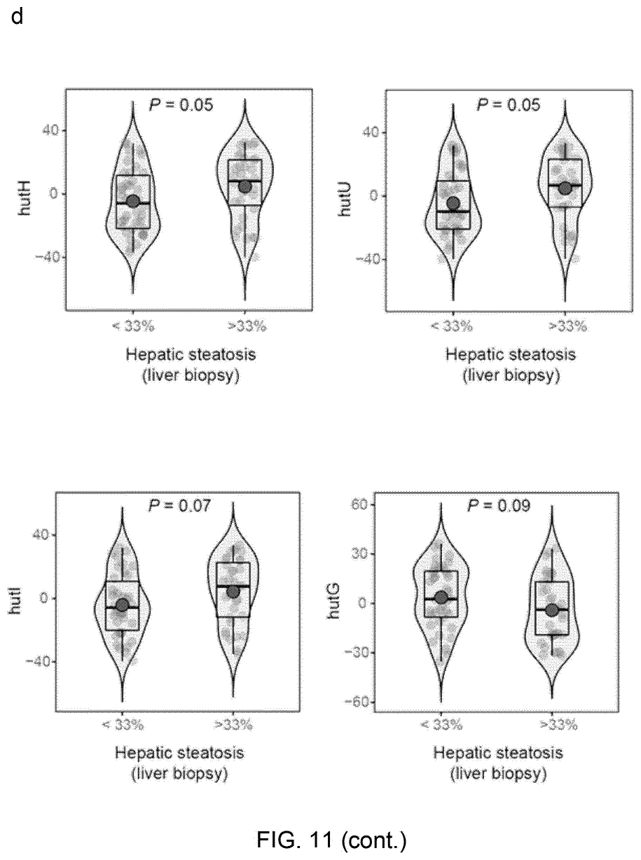

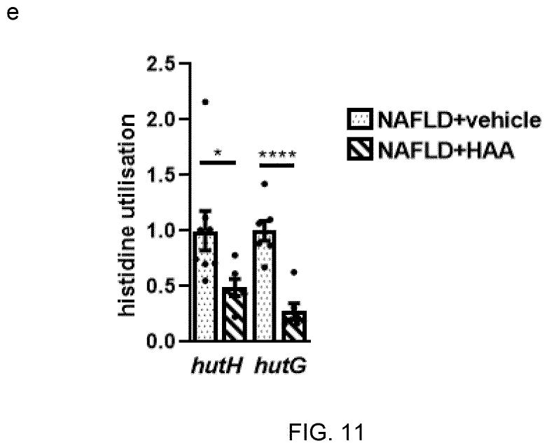

The gut microbiome plays a critical role in the pathophysiology of non-alcoholic fatty liver disease (NAFLD) (FIG. 11b) and other diseases and conditions such as obesity, IBD, visceral adipose tissue, and unhealthier metabolic profile.

Xanthomonadaceae, Rickettsiaceae, and Alteromonadales, families from the phylum Proteobacteria, are increased in hepatic steatosis. At the genus level, Bilophila and Campylobacter have also been described to be increased in NAFLD.

Histidine can be used by many bacteria and is an important carbon, nitrogen, and/or energy source for Proteobacteria. The histidine utilisation (Hut) pathway involves the elimination of ammonia from histidine to produce urocanate, imidazolone propionate and formiminoglutamate. (FIG. 11c)

To assess the potential role of the gut microbiota in modulating plasma histidine levels, a shotgun metagenomics analysis of 73 faecal samples of patients from the discovery cohort was performed. The analysis of the composition of the microbiome with bias correction (ANCOM-BC) was applied to consider the compositional nature of the metagenomic datasets and to identify differential abundant taxa associated with the circulating histidine levels controlling for age, BMI, sex, and country.

A strong negative association was identified between histidine levels and members of the phylum Proteobacteria. Families (FIG. 11a) from the phylum Proteobacteria were also strongly associated with the degree of hepatic steatosis. Circulating histidine levels were also strongly positively associated with Cyanobacteria and orders Corynebacteriales (phylum Actinobacteria) and Marinilabiliales (phylum Bacteroidetes) (FIG. 11a).

Circulating histidine was as well positively associated with bacterial indicators of a healthy gut known to produce anti-inflammatory metabolites such as species from Faecalibacterium, Bifidobacterium and Odoribacter (FIG. 11a).

In line with these results, it was found that patients with higher steatosis degree had increased levels of microbial Hut genes, which decreased after HAA supplementation (“HAA” as used herein, corresponding to the composition used in the previous examples comprising histidine, cysteine, serine and carnosine), indicating an effect of HAA administration on the associated dysbiosis.

Patients with a steatosis degree<33% and those with a>33% of liver steatosis were found to have higher abundances of microbial genes involved in the first three histidine degradation steps, which appear to be universal and involve the conversion of histidine to urocanate, hydration of urocanate to imidazole propionate and cleavage of the imidazole ring to give formiminoglutamate (FIG. 11d)

Previous results probe that supplementation of histidine-related amino acids (HAA) improves NAFLD and decreased liver inflammation in mice.

It is herein shown that HAA supplementation in NAFLD-induced mice decreases the expression of bacterial histidine utilisation genes (hutH and hutG), which were found to be increased in patients with NAFLD (FIG. 11e)

Altogether, these results show that plasma histidine levels are negatively associated with the bacterial families dysregulated in NAFLD. This microbiome dysregulation, known as dysbiosis, can be counteracted by HAA administration.

Further aspects/embodiments of the present invention can be found in the following clauses:

1.—A pharmaceutical or dietary supplement composition comprising, together with one or more pharmaceutically or nutraceutical acceptable excipients or carriers, a therapeutically or nutraceutical effective amount of at least three of: histidine or a pharmaceutically or nutraceutical acceptable salt thereof or a solvate of any of both; cysteine or a pharmaceutically or nutraceutical acceptable salt thereof or a solvate of any of both, serine or a pharmaceutically or nutraceutical acceptable salt thereof or a solvate of any of both, and carnosine or a pharmaceutically or nutraceutical acceptable salt thereof or a solvate of any of both; and/or a therapeutically or nutraceutical effective amount of a peptide, said peptide comprising a sequence of amino acids including at least three of the histidine, cysteine, serine and carnosine.

2.—The composition according to clause 1, comprising, together with one or more pharmaceutically or nutraceutical acceptable excipients or carriers, a therapeutically or nutraceutical effective amount of at least three of: histidine or a pharmaceutically or nutraceutical acceptable salt thereof or a solvate of any of both; cysteine or a pharmaceutically or nutraceutical acceptable salt thereof or a solvate of any of both, serine or a pharmaceutically or nutraceutical acceptable salt thereof or a solvate of any of both, and carnosine or a pharmaceutically or nutraceutical acceptable salt thereof or a solvate of any of both.

3.—The composition according to any one of clauses 1-2, wherein the therapeutically or nutraceutical effective amount of histidine or a pharmaceutically or nutraceutical acceptable salt thereof or a solvate of any of both is that giving a dose from 10 mg to 250 mg per Kg of the subject; the therapeutically effective amount of cysteine or a pharmaceutically or nutraceutical acceptable salt thereof or a solvate of any of both is that giving a dose from 30 to 550 mg per Kg of the subject; the therapeutically effective amount of serine or a pharmaceutically or nutraceutical acceptable salt thereof or a solvate of any of both is that giving a dose from 10 to 250 mg per Kg of the subject; and the therapeutically effective amount of carnosine or a pharmaceutically or nutraceutical acceptable salt thereof or a solvate of any of both is that giving a dose from 10 to 200 mg per Kg of the subject.

4.—The composition according to any one of clauses 1-3, comprising a therapeutically or nutraceutical effective amount of at least three of L-histidine or a pharmaceutically or nutraceutical acceptable salt thereof or a solvate of any of both; L-cysteine or a pharmaceutically or nutraceutical acceptable salt thereof or a solvate of any of both, L-serine or a pharmaceutically or nutraceutical acceptable salt thereof or a solvate of any of both, and L-carnosine or a pharmaceutically or nutraceutical acceptable salt thereof or a solvate of any of both.

5.—The composition according to any one of clauses 1-4, wherein the pharmaceutically or nutraceutical acceptable salt of histidine and of cysteine is the hydrochloride salt of L-histidine and the hydrochloride salt of L-cysteine.

6.—The composition according to any one of clauses 1-5, wherein the pharmaceutically acceptable salt of histidine is L-histidine monohydrochloride monohydrate.

7.—The composition according to any one clauses 1-6, which comprises a therapeutically or nutraceutical effective amount of histidine or a pharmaceutically or nutraceutical acceptable salt thereof or a solvate of any of both; a therapeutically or nutraceutical effective amount of cysteine or a pharmaceutically or nutraceutical acceptable salt thereof or a solvate of any of both; a therapeutically or nutraceutical effective amount of serine or a pharmaceutically or nutraceutical acceptable salt thereof or a solvate of any of both; and a therapeutically or nutraceutical effective amount carnosine or a pharmaceutically or nutraceutical acceptable salt thereof or a solvate of any of both, together with one or more pharmaceutically or nutraceutical acceptable excipients or carriers.

8.—The composition according to clause 7, comprising:
a therapeutically or nutraceutical effective amount of L-histidine monohydrochloride monohydrate;
a therapeutically or nutraceutical effective amount of L-cysteine hydrochloride;
a therapeutically or nutraceutical effective amount of L-serine; and
a therapeutically or nutraceutical effective amount of L-carnosine, together with one or more pharmaceutically or nutraceutical acceptable excipients or carriers.

9.-A food product in solid or liquid form comprising the pharmaceutical or dietary supplement composition as defined in any one of clauses 1-8.

10.—The food product according to clause 9, which is a beverage.

11.-A pharmaceutical or dietary supplement composition as defined in any one of claims of clauses 1-8, or a food product as defined in any of clauses 9-10, for use in therapy.

12.—The pharmaceutical or dietary supplement composition or the food product for use according to clause 11, which is for use in the prevention and/or treatment of a disease selected from the group consisting of a fatty liver disease, obesity, atherosclerosis and combinations thereof.

13.—The pharmaceutical or dietary supplement composition or the food product for use according to any one of clauses 11-12, which is for use in the prevention and/or treatment of a fatty liver disease selected from non-alcoholic fatty liver disease and alcoholic fatty liver disease.

14.—The pharmaceutical or dietary supplement composition or the food product for use according to clause 13, wherein the non-alcoholic fatty liver disease is selected from non-alcoholic fatty liver (NAFLD) with a simple steatosis and non-alcoholic steatohepatitis (NASH).

### CITATION LIST

Koebele, S. V. & Bimonte-Nelson, H. A. “Modeling menopause: The utility of rodents in translational behavioral endocrinology research”. Maturitas 2016, 87, 5-17.
Mardinoglu et al., “Personal model-assisted identification of NAD+ and glutathione metabolism as intervention target in NAFLD”, Mol Syst Biol-2017, vol. no. 13:916
Mong et al., “Histidine and carnosine alleviated hepatic steatosis in mice consumed high saturated fat diet”, European Journal of Pharmacology 653 (2011) 82-88
Zhao, Z.; Fu, C.; Zhang, Y.; Fu, A. Dimeric Histidine as a Novel Free Radical Scavenger Alleviates Non-Alcoholic Liver Injury. Antioxidants 2021, 10, 1529. https://doi.org/10.3390/antiox10101529

What is claimed is:

1. A pharmaceutical or dietary supplement composition comprising, together with one or more pharmaceutically or nutraceutical acceptable excipients or carriers:
(i) a therapeutically or nutraceutical effective amount of at least three of: histidine or a pharmaceutically or nutraceutical acceptable salt thereof or a solvate of any of both, cysteine or a pharmaceutically or nutraceutical acceptable salt thereof or a solvate of any of both, serine or a pharmaceutically or nutraceutical acceptable salt thereof or a solvate of any of both, and carnosine or a pharmaceutically or nutraceutical acceptable salt thereof or a solvate of any of both; and/or a therapeutically or nutraceutical effective amount of a peptide, said peptide comprising a sequence of amino acids including at least three of histidine, cysteine, serine and carnosine; or, alternatively,
(ii) a therapeutically or nutraceutical effective amount of of at least three of: histidine or a pharmaceutically or nutraceutical acceptable salt thereof or a solvate of any of both, N-acetylcysteine (NAC) or a pharmaceutically or nutraceutical acceptable salt thereof or a solvate of any of both, serine or a pharmaceutically or nutraceutical acceptable salt thereof or a solvate of any of both, and carnosine or a pharmaceutically or nutraceutical acceptable salt thereof or a solvate of any of both; and/or a therapeutically or nutraceutical effective amount of a peptide, said peptide comprising a sequence of amino acids including at least three of histidine, NAC serine and carnosine.

2. The composition according to claim 1, comprising, together with one or more pharmaceutically or nutraceutical acceptable excipients or carriers:
(i) a therapeutically or nutraceutical effective amount of histidine or a pharmaceutically or nutraceutical acceptable salt thereof or a solvate of any of both, a therapeutically or nutraceutical effective amount of cysteine or a pharmaceutically or nutraceutical acceptable salt thereof or a solvate of any of both, a therapeutically or nutraceutical effective amount of serine or a pharmaceutically or nutraceutical acceptable salt thereof or a solvate of any of both and a therapeutically or nutraceutical effective amount carnosine or a pharmaceutically or nutraceutical acceptable salt thereof or a solvate of any of both; and/or a therapeutically or nutraceutical effective amount of a peptide, said peptide comprising a sequence of amino acids including histidine, cysteine, serine and carnosine; or, alternatively,
(ii) a therapeutically or nutraceutical effective amount of histidine or a pharmaceutically or nutraceutical acceptable salt thereof or a solvate of any of both, a therapeutically or nutraceutical effective amount of NAC or a pharmaceutically or nutraceutical acceptable salt thereof or a solvate of any of both, a therapeutically or nutraceutical effective amount of serine or a pharmaceutically or nutraceutical acceptable salt thereof or a solvate of any of both, and a therapeutically or nutraceutical effective amount carnosine or a pharmaceutically or nutraceutical acceptable salt thereof or a solvate of any of both; and/or a therapeutically or nutraceutical effective amount of a peptide, said peptide comprising a sequence of amino acids including histidine, NAC, serine and carnosine.

3. The composition according to claim 1, comprising, together with one or more pharmaceutically or nutraceutical acceptable excipients or carriers:
(i) a therapeutically or nutraceutical effective amount of at least three of: histidine or a pharmaceutically or nutraceutical acceptable salt thereof or a solvate of any of both; cysteine or a pharmaceutically or nutraceutical acceptable salt thereof or a solvate of any of both, serine or a pharmaceutically or nutraceutical acceptable salt thereof or a solvate of any of both, and carnosine or a pharmaceutically or nutraceutical acceptable salt thereof or a solvate of any of both; or, alternatively,
(ii) therapeutically or nutraceutical effective amount of at least three of: histidine or a pharmaceutically or nutraceutical acceptable salt thereof or a solvate of any of both, N-acetylcysteine (NAC) or a pharmaceutically or nutraceutical acceptable salt thereof or a solvate of any of both, serine or a pharmaceutically or nutraceutical acceptable salt thereof or a solvate of any of both, and carnosine or a pharmaceutically or nutraceutical acceptable salt thereof or a solvate of any of both.

4. The composition according to claim 1, comprising, together with one or more pharmaceutically or nutraceutical acceptable excipients or carriers:
(i) a therapeutically or nutraceutical effective amount of histidine or a pharmaceutically or nutraceutical acceptable salt thereof or a solvate of any of both, a therapeutically or nutraceutical effective amount of cysteine or a pharmaceutically or nutraceutical acceptable salt thereof or a solvate of any of both, a therapeutically or nutraceutical effective amount of serine or a pharmaceutically or nutraceutical acceptable salt thereof or a solvate of any of both, and a therapeutically or nutraceutical effective amount carnosine or a pharmaceutically or nutraceutical acceptable salt thereof or a solvate of any of both; or, alternatively,
(ii) a therapeutically or nutraceutical effective amount of histidine or a pharmaceutically or nutraceutical acceptable salt thereof or a solvate of any of both, a therapeutically or nutraceutical effective amount of NAC or a pharmaceutically or nutraceutical acceptable salt thereof or a solvate of any of both, a therapeutically or nutraceutical effective amount of serine or a pharmaceutically or nutraceutical acceptable salt thereof or a solvate of any of both, and a therapeutically or nutraceutical effective amount carnosine or a pharmaceutically or nutraceutical acceptable salt thereof or a solvate of any of both.

5. The composition according to claim 1, comprising, together with one or more pharmaceutically or nutraceutical acceptable excipients or carriers, a therapeutically or nutraceutical effective amount of at least three of: histidine or a pharmaceutically or nutraceutical acceptable salt thereof or a solvate of any of both, N-acetylcysteine (NAC) or a pharmaceutically or nutraceutical acceptable salt thereof or a solvate of any of both, serine or a pharmaceutically or nutraceutical acceptable salt thereof or a solvate of any of both, and carnosine or a pharmaceutically or nutraceutical acceptable salt thereof or a solvate of any of both.

6. The composition according to claim 1, comprising, together with one or more pharmaceutically or nutraceutical acceptable excipients or carriers, a therapeutically or nutraceutical effective amount of histidine or a pharmaceutically or nutraceutical acceptable salt thereof or a solvate of any of both, a therapeutically or nutraceutical effective amount of NAC or a pharmaceutically or nutraceutical acceptable salt thereof or a solvate of any of both, a therapeutically or nutraceutical effective amount of serine or a pharmaceutically or nutraceutical acceptable salt thereof or a solvate of any of both, and a therapeutically or nutraceutical effective amount carnosine or a pharmaceutically or nutraceutical acceptable salt thereof or a solvate of any of both.

7. The composition according to claim 1, wherein the therapeutically or nutraceutical effective amount of histidine or a pharmaceutically or nutraceutical acceptable salt thereof or a solvate of any of both is that giving a dose from 7.5 mg to 250 mg per Kg of the subject; the therapeutically effective amount of cysteine or a pharmaceutically or nutraceutical acceptable salt thereof or a solvate of any of both is that giving a dose from 2 to 550 mg per Kg of the subject; the therapeutically effective amount of serine or a pharmaceutically or nutraceutical acceptable salt thereof or a solvate of any of both is that giving a dose from 1.3 to 250 mg per Kg of the subject; and the therapeutically effective amount of carnosine or a pharmaceutically or nutraceutical acceptable salt thereof or a solvate of any of both is that giving a dose from 4.5 to 200 mg per Kg of the subject.

8. The composition according to claim 7, wherein the therapeutically or nutraceutical effective amount of histidine or a pharmaceutically or nutraceutical acceptable salt thereof or a solvate of any of both is that giving a dose from 10 mg to 250 mg per Kg of the subject; the therapeutically effective amount of cysteine or a pharmaceutically or nutraceutical acceptable salt thereof or a solvate of any of both is that giving a dose from 30 to 550 mg per Kg of the subject; the therapeutically effective amount of serine or a pharmaceutically or nutraceutical acceptable salt thereof or a solvate of any of both is that giving a dose from 10 to 250 mg per Kg of the subject; and the therapeutically effective amount of carnosine or a pharmaceutically or nutraceutical acceptable salt thereof or a solvate of any of both is that giving a dose from 10 to 200 mg per Kg of the subject.

9. The composition according to claim 1, wherein the therapeutically or nutraceutical effective amount of histidine or a pharmaceutically or nutraceutical acceptable salt thereof or a solvate of any of both is that giving a dose from 7.5 mg to 250 mg per Kg of the subject; the therapeutically effective amount of NAC or a pharmaceutically or nutraceutical acceptable salt thereof or a solvate of any of both is that giving a dose from 2 to 550 mg per Kg of the subject; the therapeutically effective amount of serine or a pharmaceutically or nutraceutical acceptable salt thereof or a solvate of any of both is that giving a dose from 1.3 to 250 mg per Kg of the subject; and the therapeutically effective amount of carnosine or a pharmaceutically or nutraceutical acceptable salt thereof or a solvate of any of both is that giving a dose from 4.5 to 200 mg per Kg of the subject.

10. The composition according to claim 9, wherein the therapeutically or nutraceutical effective amount of histidine or a pharmaceutically or nutraceutical acceptable salt thereof or a solvate of any of both is that giving a dose from 10 mg to 250 mg per Kg of the subject; the therapeutically effective amount of NAC or a pharmaceutically or nutraceutical acceptable salt thereof or a solvate of any of both is that giving a dose from 30 to 550 mg per Kg of the subject; the therapeutically effective amount of serine or a pharmaceutically or nutraceutical acceptable salt thereof or a solvate of any of both is that giving a dose from 10 to 250 mg per Kg of the subject; and the therapeutically effective amount of carnosine or a pharmaceutically or nutraceutical acceptable salt thereof or a solvate of any of both is that giving a dose from 10 to 200 mg per Kg of the subject.

11. The composition according to claim 1, wherein the pharmaceutically or nutraceutical acceptable salt of histidine is the hydrochloride salt of L-histidine.

12. The composition according to claim 1, wherein the pharmaceutically acceptable salt of histidine is L-histidine monohydrochloride monohydrate.

13. The composition according to claim 1, wherein the pharmaceutically or nutraceutical acceptable salt of cysteine is the hydrochloride salt of L-cysteine.

14. The composition according to claim 1, comprising:
a therapeutically or nutraceutical effective amount of L-histidine monohydrochloride monohydrate;
a therapeutically or nutraceutical effective amount of NAC or a therapeutically or nutraceutical effective amount of L-cysteine hydrochloride;
a therapeutically or nutraceutical effective amount of L-serine; and
a therapeutically or nutraceutical effective amount of L-carnosine,
together with one or more pharmaceutically or nutraceutical acceptable excipients or carriers.

15. (canceled)

16. A food product in solid or liquid form comprising the pharmaceutical or dietary supplement composition as defined in claim 1.

17. The food product according to claim 16, which is a beverage.

18-23. (canceled)

24. A method for the prevention and/or treatment of a disorder or disease selected from the group consisting of a fatty liver disease, obesity, overweight, atherosclerosis, dysbiosis, and combinations thereof, comprising administering a therapeutically or nutraceutical effective amount of the composition as defined in claim 1, together with pharmaceutically or nutraceutical acceptable excipients and/or carriers, in a subject in need thereof.

25. The method for the prevention and/or treatment of a disorder or disease according to claim 24, wherein the disorder or disease is a fatty liver disease selected from non-alcoholic fatty liver disease and alcoholic fatty liver disease.

26. The method for the prevention and/or treatment of a disorder or disease according to claim 25, wherein the non-alcoholic fatty liver disease is selected from non-alcoholic fatty liver (NAFLD) with a simple steatosis and non-alcoholic steatohepatitis (NASH).

27. A method for the prevention and/or treatment of menopause-associated symptoms, comprising administering a therapeutically or nutraceutical effective amount of the composition according to claim 1, together with pharmaceutically or nutraceutical acceptable excipients and/or carriers, in a female mammal in need thereof.
